(19) United States
(12) Patent Application Publication
(10) Pub. No.: US 2026/0207352 A1
(43) Pub. Date: Jul. 23, 2026

(54) PROSTHETIC DEVICES FOR BONE IMPLANTS

(71) Applicant: Point Designs, Inc., Austin, TX (US)

(72) Inventors: Benjamin Pulver, Louisville, CO (US); Serena Kishek, Lakewood, CO (US); Levin Sliker, Boulder, CO (US); Richard Weir, Lafayette, CO (US)

(73) Assignee: Point Designs, Inc., Austin, TX (US)

(21) Appl. No.: 19/451,695

(22) Filed: Jan. 16, 2026

Related U.S. Application Data
(60) Provisional application No. 63/746,735, filed on Jan. 17, 2025.

(51) Int. Cl. A61F 2/58; A61F 2/50; A61F 2/68; A61F 2/78

(52) U.S. Cl. CPC A61F 2/586; A61F 2/68; A61F 2002/5072; A61F 2002/6854; A61F 2002/7887

(57) ABSTRACT

A prosthetic finger can include a hub that attaches to a bone implant, a first phalange coupled to the hub, and a second phalange coupled to the first phalange. The first phalange can move in at least one of flexion or extension relative to the hub. The prosthetic finger can include a lever coupled to the first phalange. The lever can move between a first position and a second position. In the first position, the lever can prevent movement of the first phalange and the second phalange in extension. In the second position, the lever can allow movement of the first phalange and the second phalange in extension.

### CROSS-REFERENCE TO RELATED PATENT APPLICATIONS

This application claims the benefit of and priority to U.S. Provisional Patent Application No. 63/746,735 filed Jan. 17, 2025, the entirety of which is incorporated herein by reference.

### FIELD

The present disclosure relates generally to prosthetic devices. More particularly, the present disclosure relates to prosthetic devices for the hand.

### BACKGROUND

Finger or partial hand prostheses can be used to restore function and/or appearance of a missing portion of a finger or a thumb. A bone implant may be used to attach a prosthetic finger and/or hand prosthesis to a user. A bone implant can be anchored to a bone of a person and a portion of the bone implant may extend through the person's skin. Traditional finger prostheses may have no motion or a limited range of motion when attached to a bone implant.

### SUMMARY

At least one aspect relates to a prosthetic finger including a hub that attaches to a bone implant. The prosthetic finger can include a first phalange coupled to the hub and configured to move in at least one of flexion or extension relative to the hub. The prosthetic finger can include a second phalange coupled to the first phalange. The prosthetic finger can include a lever coupled to the first phalange and configured to move between a first position and a second position. In the first position, the lever can prevent movement of the first phalange and the second phalange in extension. In the second position, the lever can allow movement of the first phalange and the second phalange in extension.

In some implementations, the hub defines a set of teeth. In the first position, the lever can engage with the set of teeth to prevent movement of the first phalange and the second phalange in extension. In the second position, the lever can disengage with the set of teeth to allow movement of the first phalange and the second phalange in extension. In some implementations, the prosthetic finger includes a stop coupled to the hub and a prop coupled to the first phalange. The prop can contact the stop and can move the lever from the first position to the second position. In some implementations, the prosthetic finger includes a spring coupled to at least one of the first phalange or the second phalange. The spring can provide a biasing force that urges the first phalange and the second phalange to move in extension when the lever is in the second position. In some implementations, the prosthetic finger includes a spring coupled to the first phalange. The spring can provide a biasing force that urges the lever into the first position. In some implementations, the prosthetic finger includes a collar coupled to the hub and the bone implant. The hub can clamp the collar to the bone implant. In some implementations, the hub includes a slot, a first protrusion extending outward from the hub and positioned on a first side of the slot, and a second protrusion extending outward from the hub and positioned on a second side of the slot. The slot, the first protrusion, and the second protrusion can facilitate clamping of the collar to the bone implant. In some implementations, the prosthetic finger includes a link bar coupled to the hub and the second phalange. The link bar can couple motion of the first phalange to motion of the second phalange such that, when the first phalange is moved in flexion, the second phalange is also moved in flexion.

Another aspect relates to a prosthetic finger including a first phalange configured to move in at least one of flexion or extension. The prosthetic finger can include a second phalange coupled to the first phalange. The prosthetic finger can include a third phalange coupled to the second phalange. The prosthetic finger can include a lever coupled to the second phalange and configured to move between a first position and a second position. In the first position, the lever can allow movement of the first phalange, the second phalange, and the third phalange in flexion. In the second position, the lever can allow movement of the first phalange, the second phalange, and the third phalange in extension.

In some implementations, the prosthetic finger includes a hub coupled to the first phalange. The hub can define a set of teeth. In the first position, the lever can engage with the set of teeth to allow movement of the first phalange, the second phalange, and the third phalange in flexion. In the second position, the lever can disengage with the set of teeth to allow movement of the first phalange, the second phalange, and the third phalange in extension. The prosthetic finger can further include a stop coupled to the hub and a prop coupled to the first phalange. The prop can contact the stop and can move the lever from the first position to the second position. In some implementations, the prosthetic finger further includes a collar and a bone implant. The collar can be coupled to the hub and the bone implant. The hub can clamp the collar to the bone implant. In some implementations, the prosthetic finger includes a link bar coupled to the hub and the second phalange and a second link bar coupled to the first phalange and the third phalange. The link bar can couple motion of the first phalange to motion of the second phalange. The second link bar can couple motion of the first phalange to motion of the third phalange. In some implementations, the prosthetic finger includes a spring coupled to at least one of the first phalange or the second phalange. The spring can provide a biasing force that urges the first phalange, the second phalange, and the third phalange to move in extension when the lever is in the second position. In some implementations, the prosthetic finger includes a spring coupled to the first phalange. The spring can provide a biasing force that urges the lever into the first position.

Another aspect relates to a prosthetic finger including a track that attaches to a bone implant. The prosthetic finger can include a first phalange coupled to the track. The first phalange can move in at least one of flexion or extension along the track. The prosthetic finger can include a second phalange coupled to the first phalange. The prosthetic finger can include a lever coupled to the first phalange and configured to move between a first position and a second position. In the first position, the lever can prevent movement of the first phalange and the second phalange in extension. In the second position, the lever can allow movement of the first phalange and the second phalange in extension.

In some implementations, the track defines a set of teeth. In the first position, the lever can engage with the set of teeth to prevent movement of the first phalange and the second phalange in extension. In the second position, the lever can disengage with the set of teeth to allow movement of the first phalange and the second phalange in extension. In the first position, the lever can allow movement of the first phalange and the second phalange in flexion. In some implementations, the prosthetic finger includes a link bar coupled to the track and the second phalange. The link bar can couple motion of the first phalange to motion of the second phalange such that, when the first phalange is moved in flexion, the second phalange is also moved in flexion. In some implementations, the prosthetic finger further includes a stop coupled to the track and a prop coupled to the first phalange. The prop can contact the stop and can move the lever from the first position to the second position.

These and other aspects and implementations are discussed in detail below. The foregoing information and the following detailed description include illustrative examples of various aspects and implementations and provide an overview or framework for understanding the nature and character of the claimed aspects and implementations. The drawings provide illustration and a further understanding of the various aspects and implementations and are incorporated in and constitute a part of this specification. Aspects can be combined, and it will be readily appreciated that features described in the context of one aspect can be combined with other aspects.

### BRIEF DESCRIPTION OF THE DRAWINGS

The disclosure will be readily understood by the following detailed description in conjunction with the accompanying drawings, wherein like reference numerals designate like elements, and in which:

FIG. 1 shows an example bone implant, in accordance with one or more implementations;

FIG. 2 shows an exploded view of the bone implant of FIG. 1, in accordance with one or more implementations;

FIG. 3 shows a perspective view of a prosthetic finger that attaches to the bone implant of FIG. 1, in accordance with one or more implementations;

FIG. 4 shows another perspective view of the prosthetic finger of FIG. 3, in accordance with one or more implementations;

FIG. 5 shows an exploded view of the prosthetic finger shown in FIGS. 3 and 4, in accordance with one or more implementations;

FIG. 6 shows an example of a hub and collar for attaching a prosthetic finger to the bone implant of FIG. 1, in accordance with one or more implementations;

FIG. 7 shows a portion of the prosthetic finger of FIG. 3, in accordance with one or more implementations;

FIG. 8 shows an example of a locking mechanism for a prosthetic finger, in accordance with one or more implementations;

FIG. 9 shows a perspective view of a prosthetic finger that attaches to the bone implant of FIG. 1, in accordance with one or more implementations;

FIG. 10 shows another perspective view of the prosthetic finger of FIG. 9, in accordance with one or more implementations;

FIG. 11 shows an exploded view of the prosthetic finger of FIG. 9 and the bone implant of FIG. 1, in accordance with one or more implementations

FIG. 12 show an exploded view of the prosthetic finger of FIG. 9, in accordance with one or more implementations;

FIG. 13 shows a perspective view of a prosthetic finger that attaches to a bone implant, in accordance with one or more implementations;

FIG. 14 shows another perspective view of the prosthetic finger of FIG. 13, in accordance with one or more implementations;

FIG. 15 shows an exploded view of the prosthetic finger shown in FIG. 13, in accordance with one or more implementations;

FIG. 16 shows an example of a track of the prosthetic finger of FIG. 13, in accordance with one or more implementations;

FIG. 17 shows a perspective view of a prosthetic finger that attaches to the bone implant of FIG. 1, in accordance with one or more implementations;

FIG. 18 shows a portion of the prosthetic finger of FIG. 17, in accordance with one or more implementations;

FIG. 19 shows an exploded view of the prosthetic finger of FIG. 17, in accordance with one or more implementations;

FIG. 20 shows another portion of the prosthetic finger of FIG. 17, in accordance with one or more implementations;

FIG. 21 shows an example of a hub and collar for attaching a prosthetic finger to the bone implant of FIG. 1, in accordance with one or more implementations;

FIG. 22 shows a perspective view of a prosthetic finger, in accordance with one or more implementations;

FIG. 23 shows a portion of the prosthetic finger of FIG. 22, in accordance with one or more implementations;

FIG. 24 shows another portion of the prosthetic finger of FIG. 22, in accordance with one or more implementations;

FIG. 25 shows a perspective view of a hand including a plurality of prosthetic fingers, in accordance with one or more implementations;

FIG. 26 shows a perspective view of a hub for a prosthetic finger, in accordance with one or more implementations;

FIG. 27 shows a portion of the hub of FIG. 26, in accordance with one or more implementations;

FIG. 28 shows another portion of the hub of FIG. 26, in accordance with one or more implementations; and

FIG. 29 shows an example of a hub for a prosthetic finger, in accordance with one or more implementations.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various implementations described herein and, accordingly, may not necessarily be presented or illustrated to scale, and are not intended to indicate any preference or requirement for an illustrated implementation.

### DETAILED DESCRIPTION

Below are detailed descriptions of various concepts related to, and approaches, methods, apparatuses, and systems for implementing the various techniques described herein. The various concepts introduced above and discussed in greater detail below may be implemented in any of numerous ways, as the described concepts are not limited to any particular manner of implementation. Examples of specific implementations and applications are provided primarily for illustrative purposes.

### I. Overview

Implementations described herein are directed to a prosthetic finger that attaches to a bone implant. The prosthetic finger can move in extension and flexion to help restore function of a user's hand (e.g., gripping, pointing, etc.). The type of attachment mechanism for a prosthesis (e.g., bone implant, socket attachment, etc.) may be determined by a variety of clinical factors, which may include, for example and without limitation, an amputation location, type of injury, extent of injury, or an elective surgery. In some cases, the type of prosthetic finger or function of the prosthetic finger may be a secondary consideration for selecting a type of attachment mechanism. For example, other clinical factors such as a location of the attachment mechanism may be a driving factor when selecting the type of attachment mechanism. In some cases, a bone implant can be used as an attachment mechanism. The bone implant can include a threaded anchor that is inserted into the bone and an abutment which is attached to the threaded anchor and extends through the skin to provide an attachment point for the prosthetic finger. Once placed, the bone implant can be a permanent fixture, and may only be removed if medically necessary.

The implementations described herein are directed to a functional prosthetic finger that can attach to a bone implant. The prosthetic finger can move in extension, flexion, and/or lock in a selected position, as described herein. As used herein, the term “extension” refers to movement of the base of the finger away from the palm of a hand. When a finger is substantially extended, a length of the bones may be roughly aligned, although some deviation from this alignment may occur. As used herein, the term “flexion” refers to movement of the base of the finger toward the palm of the hand. When a finger is substantially flexed, the finger may be curled toward the palm such as used in a grasping motion.

The prosthetic finger can include structures that couple the prosthetic finger to a bone implant, while maintaining functionality of the prosthetic finger to move in flexion, extension and/or retain a selected position. The structures can couple the prosthetic finger to a portion of an abutment that extends through the skin and is located outside of a user's body. In some implementations, the prosthetic finger can include a hub that attaches to the abutment, as described herein. In other implementations, the structures can be integrated with a phalange of the prosthetic finger and the phalange can attach to the abutment. In further implementations, the prosthetic finger can include a track that attaches to the abutment. These structures can include components that allow the alignment of the prosthetic finger with respect to the user to be adjusted. For example, the structures can allow the prosthetic finger to be rotated with respect to a fixed abutment, thereby allowing the alignment of the prosthetic finger with respect to the body (e.g., the hand and other fingers) to be adjusted.

In some implementations, the prosthetic finger includes features that allow the prosthetic finger to be removed and reattached from the bone implant by a user and/or clinician. Accordingly, the bone implant may be effectively considered a permanent implant and the prosthetic finger may be a removable device that is positioned adjacent to a skin surface and outside the body.

The examples provided herein are described primarily in the context of two-phalange prosthetic fingers and three-phalange prosthetic fingers. However, these concepts can be applied to prosthetic fingers having a single phalange, for example, a prosthetic finger having a single phalange coupled to a hub or track, as described herein.

The term “prosthetic finger” is used herein to refer to any of the digits of a hand including a finger and/or a thumb. That is, the prosthetic finger described herein may be used to restore function to any one of the fingers and/or a thumb. Additionally, the concepts described herein may be applied to other types of prostheses that are used at other portions of a user's body. For example, the concepts described herein may be applied to a prosthetic hand where the hand is attached to a bone implant located in the vicinity of a wrist joint of a user. In other cases, the concepts described herein can be applied to a prothesis attached to a user along other portions of the user's upper limb or at other locations of the body.

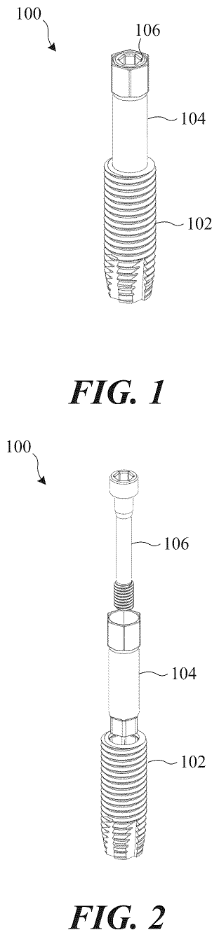

These and other implementations are discussed below with reference to FIGS. 1-29. However, those skilled in the art will readily appreciate that the detailed description given herein with respect to these Figures is for explanatory purposes only and should not be construed as limiting.

### II. Overview of Prosthetic Finger

Referring to FIGS. 1 and 2, an example bone implant 100 is shown. FIG. 1 shows an example of the bone implant 100 assembled, which is the configuration of the bone implant after it has been attached to a user. FIG. 2 shows an exploded view of the bone implant 100, for illustrative purposes. The bone implant 100 can include an anchor 102, an abutment 104, and a connector 106. The anchor 102 attaches to a bone(s) of the user. For example, the anchor 102 may include threads that are screwed into the bone. The anchor 102 can be coupled to the bone using any suitable method including other anchoring features, adhesives, and so on. The anchor 102 can include an aperture (e.g., an opening, a passage, etc.) and the abutment 104 can be received in the aperture of the anchor 102. The abutment 104 can also include an aperture (e.g., an opening, a passage, etc.) and the connector 106 can be received in the aperture of the abutment 104 to couple the abutment 104 to the anchor 102. In some implementations, the connector 106 includes a threaded fastener to couple the abutment 104 to the anchor 102, although other connector 106 configurations are possible.

The abutment 104 can include a first portion that interfaces with the anchor 102 and is positioned within the user's body and a second portion that is positioned outside the user's body. Once installed, the abutment 104 may effectively be considered a permanent implant (e.g., only removed if medically necessary), as the skin and/or other tissue may heal to and/or around a portion of the abutment 104.

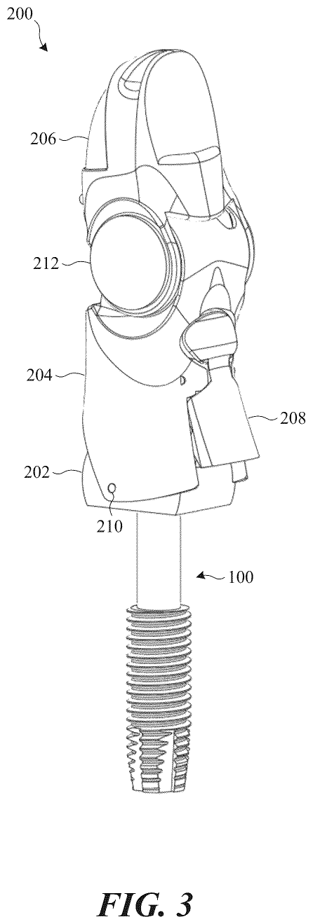

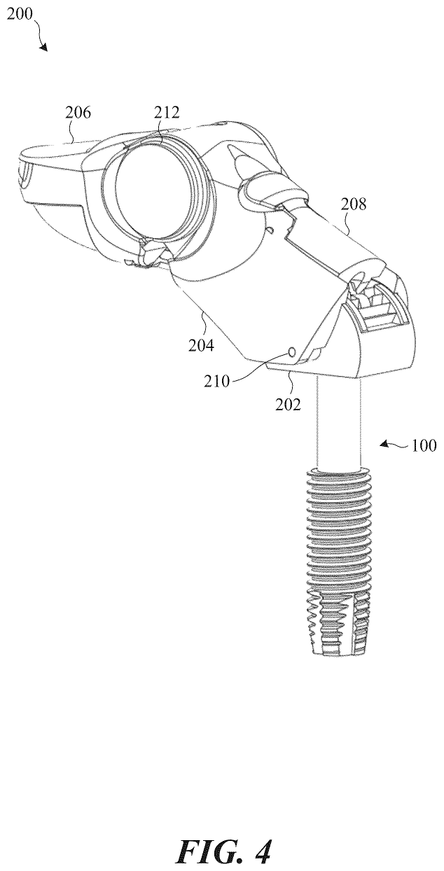

Referring now to FIGS. 3 and 4, an example prosthetic finger 200 that attaches to the bone implant 100, is shown. FIG. 3 shows an example of the prosthetic finger 200 in an extended configuration and FIG. 4 shows an example of the prosthetic finger 200 in a partially flexed configuration (e.g., flexion of the prosthetic finger 200 from a more extended configuration).

The prosthetic finger 200 can include a hub 202, a first phalange 204, a second phalange 206, and a lever 208. As described in more detail herein, the hub 202 can be coupled to the bone implant 100 (e.g., at the abutment 104). The first phalange 204 can have a first end coupled to the hub 202 and can move in flexion and extension relative to the hub 202. For example, the prosthetic finger 200 can include one or more rotational structures (e.g., bearings, pins, etc.), shown as pin 210 (e.g., shafts, bars, etc.), that pivotally couples the first phalange 204 to the hub 202 and allows the first phalange 204 to rotate with respect to the hub 202. The second phalange 206 can be coupled to the first phalange 204 and can move in flexion and extension relative to the first phalange 204 and/or the hub 202. For example, the prosthetic finger 200 can include one or more rotational structures (e.g., bearings, pins, etc.), shown as pin 212 (e.g., shaft, bar, etc.), that pivotally couples the second phalange 206 to the first phalange 204 and allows the second phalange 206 to rotate with respect to the first phalange 204.

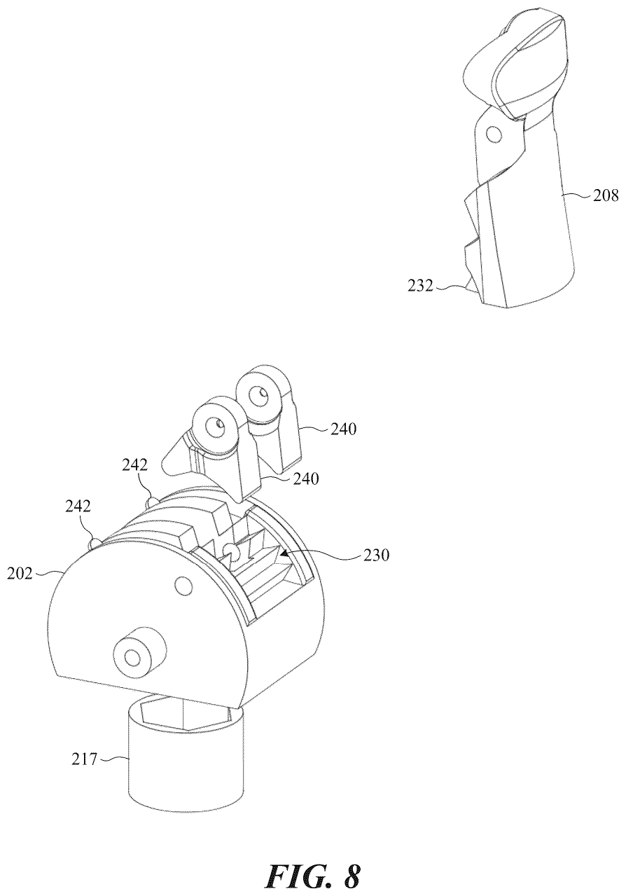

The lever 208 can prevent movement of the first phalange 204 and/or the second phalange 206 in extension and/or flexion. The lever 208 can be coupled to the first phalange 204 via a pin 214 (e.g., a shaft, a bar, etc.). The pin 214 may pivotally couple the lever 208 to the first phalange 204 such that the lever 208 can move (e.g., rotate, pivot, etc.) between a first position and a second position. In the first position, the lever 208 can engage with the hub 202 (e.g., by engaging with a set of teeth 230, such as discussed with respect to FIG. 8) to prevent movement of the first phalange 204 and the second phalange 206 in extension while allowing movement of the first phalange 204 and the second phalange 206 in flexion. For example, the prosthetic finger 200 can be a passive prosthetic finger in which a user moves the first phalange 204 and/or the second phalange 206 to a desired position by manually causing the first phalange 204 and/or the second phalange 206 to move in flexion. As the prosthetic finger 200 is moved in flexion, the lever 208 is in the first position and engages with the hub 202 to prevent movement of the prosthetic finger 200 in extension.

In the second position, the lever 208 can disengage with the hub 202 (e.g., disengages with the set of teeth 230, discussed with respect to FIG. 8) to allow movement of the first phalange 204 and/or the second phalange 206 in extension and flexion (e.g., free movement of the prosthetic finger 200). In some implementations, the prosthetic finger 200 includes a first spring element 215 (e.g., a spring, a coil spring, etc.) that biases the lever 208 in the first position. The first spring element 215 can be coupled to the first phalange 204 and can provide a biasing force that urges the lever 208 into the first position. A user input can actuate the lever 208 to the second position, for example, by rotating/pivoting the lever 208 about the pin 214 to disengage the lever 208 from the hub 202 and allow movement of the first phalange 204 and/or the second phalange 206 in extension and flexion.

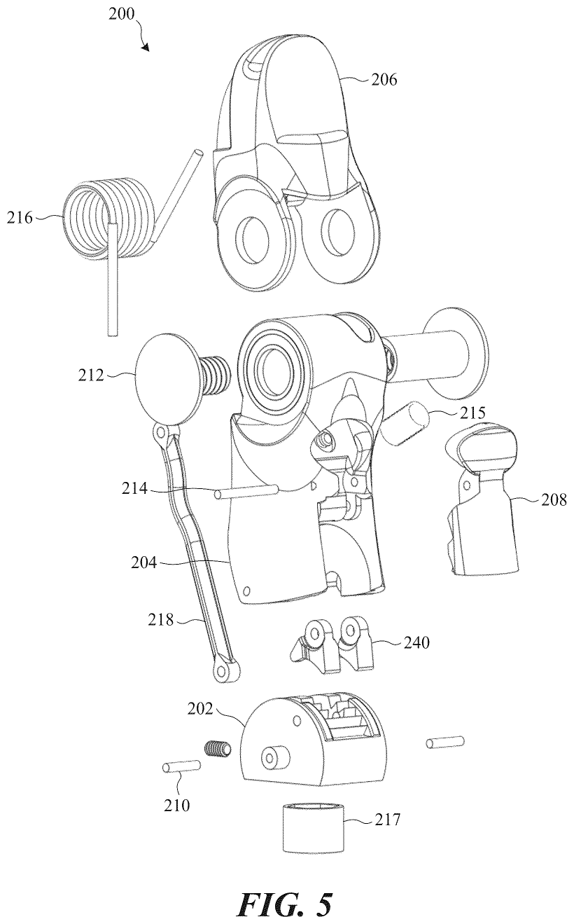

Referring now to FIG. 5, an exploded view of the prosthetic finger 200 shown in FIGS. 3 and 4, is shown. As shown in FIG. 5, the prosthetic finger 200 can include a second spring element 216 (e.g., a spring, a spring element, a torsional spring, etc.) coupled to the first phalange 204 and/or the second phalange 206. In various implementations, the second spring element 216 is coupled to the pin 212 (e.g., wound about). The second spring element 216 can provide a biasing force that urges the first phalange 204 and the second phalange 206 to move in extension. For example, when the lever 208 is in the second position (e.g., disengaged from the hub 202) the second spring element 216 can cause the first phalange 204 and the second phalange 206 to move in extension. When the lever 208 is in the first position, the engagement of the lever 208 with the hub 202 (e.g., the set of teeth 230) can prevent movement in extension and the second spring element 216 may exert a force on the lever 208 and the hub 202.

The prosthetic finger 200 can also include a collar 217 coupled to the hub 202 (e.g., received within the hub 202). As described in more detail herein, the collar 217 can attach to the bone implant 100 (e.g., to the abutment 104) to couple the hub 202 to the bone implant 100.

The prosthetic finger 200 can include a link bar 218 that couples motion of the first phalange 204 to motion of the second phalange 206. For example, the link bar 218 can cause the first phalange 204 and the second phalange 206 to both move in flexion or both move in extension. Accordingly, if a user manually moves the second phalange 206 in flexion, the link bar 218 can cause the first phalange 204 to also move in flexion. If a user manually moves the first phalange 204 in flexion, the link bar 218 can cause the second phalange 206 to also move in flexion. Accordingly, the link bar 218 can couple the movements of the first phalange 204 and the second phalange 206.

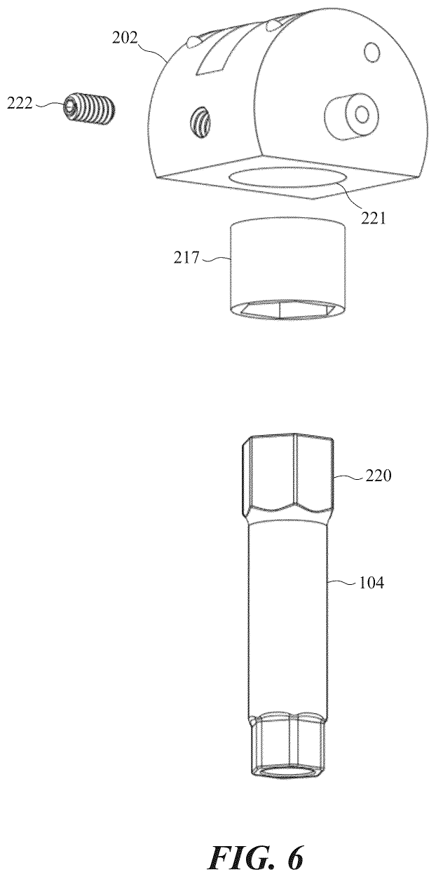

Referring now to FIG. 6, the hub 202 and the collar 217 are shown in further detail. The hub 202 and the collar 217 can be used to attach the prosthetic finger 200 to the bone implant 100. As shown in FIG. 6, the collar 217 can attach to the abutment 104. For example, the abutment 104 can include a head portion 220 which is positioned outside the body of the user when the bone implant 100 is attached. The collar 217 can rigidly attach to the abutment 104 such that there is no movement between the collar 217 and the abutment 104. For example, in some implementations, the collar 217 is press-fit onto the head portion 220 and the coupling of the collar 217 with respect to the head portion 220 is configured to prevent movement between the collar 217 and the head portion 220.

In some implementations, the collar 217 defines an opening that has geometric features that mate with geometric features of the abutment 104. For example, if the head portion 220 of the abutment 104 is hex-shaped, the collar 217 can define an opening having a hex-shaped configuration that mates with the head portion 220. Additionally or alternatively, the collar 217 can be attached to the abutment 104 using other methods, such as the collar 217 and the head portion 220 including threaded features, adhesive materials, welding, etc. In some implementations, the collar 217 is configured as a multipart component that clamps to the abutment 104. The clamping of the collar 217 can provide forces that prevent movement between the collar 217 and the abutment 104.

The hub 202 can couple to the collar 217 and be configured to rotate and/or translate with respect to the collar 217. For example, the hub 202 can define an opening 221 that mates with the collar 217. The collar 217 can be positioned within the opening 221 and the hub 202 can rotate and/or translate with respect to the collar 217. For example, the hub 202 can be attached to the collar 217 and rotated relative to the collar 217 such that the hub 202 is aligned relative to other fingers of the user. The prosthetic finger 200 can include a retaining mechanism that selectively prevents movement between the hub 202 and the collar 217 after alignment. For example, the retaining mechanism can include a set screw 222 that engages with the hub 202 and engages with the collar 217 to prevent movement between the hub 202 and the collar 217. Additionally or alternatively, the retaining mechanism can include threads or teeth on both the collar 217 and the hub 202 that engage with each other and prevent rotation. Accordingly, the hub 202 can be positioned on the collar 217, attached to the abutment 104, and the alignment of the hub 202 (and the prosthetic finger 200) can be adjusted with respect to the bone implant 100. Once in a desired position the retaining mechanism can be engaged to lock the hub 202 in place and prevent further movement between the bone implant 100 and the hub 202.

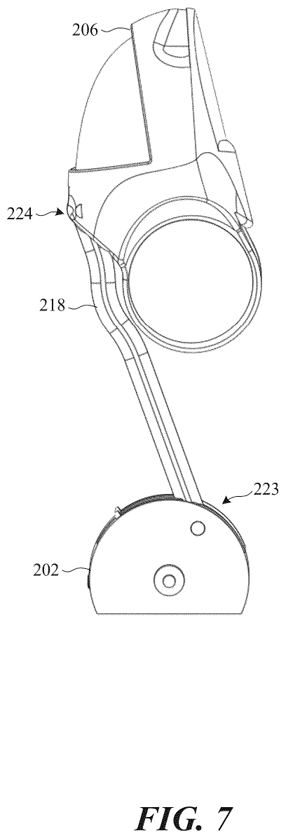

Referring now to FIG. 7, the hub 202, the link bar 218, and the second phalange 206 are shown in further detail. As shown in FIG. 7, the link bar 218 can include a first link bar end 223 coupled to the hub 202 and a second link bar end 224 coupled to the second phalange 206. The link bar 218 can rotate with respect to the hub 202 and the second phalange 206. For example, the first link bar end 223 can rotate about the hub 202 and the second link bar end 224 can rotate about the second phalange 206. In some cases, the link bar 218 can be attached to each of the hub 202 and the second phalange 206 using pins or any other suitable rotational coupling (e.g., bearings, etc.). As described herein, the link bar 218 can couple motion of the first phalange 204 and the second phalange 206.

Referring now to FIG. 8, the prosthetic finger 200 can further include a locking mechanism configured to retain the prosthetic finger 200 at a position of flexion and/or extension. The locking mechanism can include the hub 202 and the lever 208. For example, the hub 202 can define a set of teeth 230 and the lever 208 can include an engagement feature 232 that engages with the set of teeth 230 (e.g., a ratcheting mechanism) when the lever 208 is in the first position. The set of teeth 230 and the engagement feature 232 can allow movement in a first direction (e.g., flexion) and prevent movement in a second, opposite direction (e.g., extension). For example, when the lever 208 is in the first position, the engagement feature 232 can engage with the set of teeth 230 such that movement of the prosthetic finger 200 in flexion is allowed (e.g., the engagement feature 232 slides over the set of teeth 230). With the lever 208 in the first position, attempting to move the prosthetic finger 200 in extension (e.g., by manually moving the first phalange 204 and/or the second phalange 206) causes the engagement feature 232 to abut against the set of teeth 230 (e.g., one tooth of the set of teeth 230), thereby preventing movement of the prosthetic finger 200 in extension. In this way, when the lever 208 is in the first position, the lever 208 and the hub 202 allow movement of the prosthetic finger 200 in flexion while preventing movement of the prosthetic finger 200 in extension. Furthermore, engagement between the set of teeth 230 and the engagement feature 232 acts as a locking mechanism, retaining the first phalange 204 and the second phalange 206 at a desired position of flexion (e.g., partially flexed, fully flexed, etc.). When the lever 208 is rotated about the pin 214 into the second position, the engagement feature 232 disengages from the set of teeth 230 and the second spring element 216 biases the first phalange 204 and the second phalange 206 into extension.

In some implementations, the prosthetic finger 200 further includes props 240 (e.g., at least one prop, a pair of props, a prop, etc.). The props 240 can facilitate automatic return of the prosthetic finger 200 to an extended configuration (e.g., as shown in FIG. 3) in response to reaching a defined flexion point (e.g., maximum flexion, a threshold flexion value, etc.). For example, prosthetic finger 200 can include stops 242 (e.g., at least one stop, a pair of stops, a stop etc.) coupled to the hub 202. The props 240 can be coupled to the first phalange 204 and configured to contact the stops 242 at the defined flexion point. The contact of the props 240 with the stops 242 can cause the props 240 to contact the lever 208 and transition the lever 208 from the first position to the second position. When the lever 208 is in the second position, the second spring element 216 can cause the first phalange 204 and the second phalange 206 to move in extension. The props 240 can be configured such that the props 240 do not contact the lever 208 until the defined flexion point is reached, whereby the props 240 transition the lever 208 into the second position.

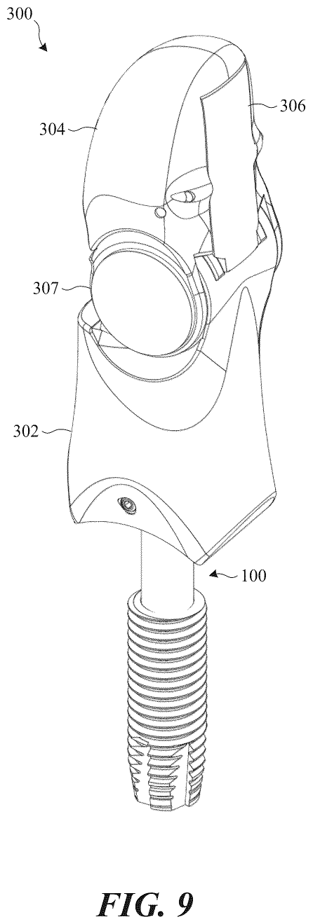

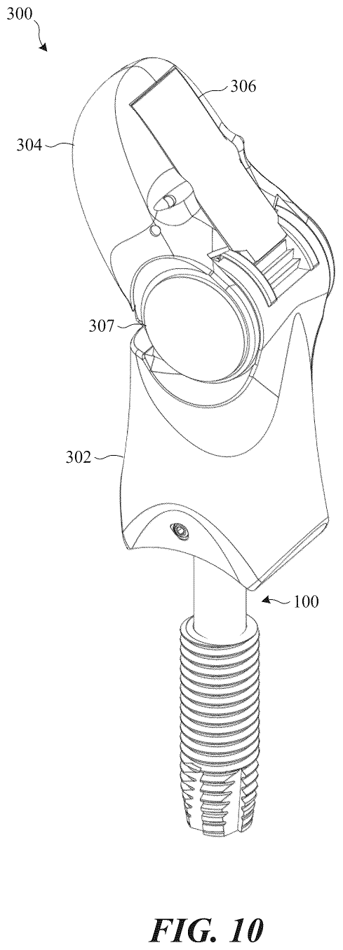

Referring now to FIGS. 9 and 10, a prosthetic finger 300 that attaches to the bone implant 100 is shown. FIG. 9 shows an example of the prosthetic finger 300 in an extended configuration and FIG. 10 shows an example of the prosthetic finger 300 in a partially flexed configuration (e.g., flexion of the prosthetic finger 300 from a more extended configuration).

The prosthetic finger 300 can include a first phalange 302, a second phalange 304 and a lever 306. The first phalange 302 can attach to the bone implant 100. The second phalange 304 can have a first end coupled to the first phalange 302 and can move in flexion and extension relative to the first phalange 302. For example, the prosthetic finger 300 can include one or more rotational structures (e.g., bearings, pins, etc.), shown as pin 307, that pivotally couples the second phalange 304 to the first phalange 302 and allows the second phalange 304 to rotate relative to the first phalange 302.

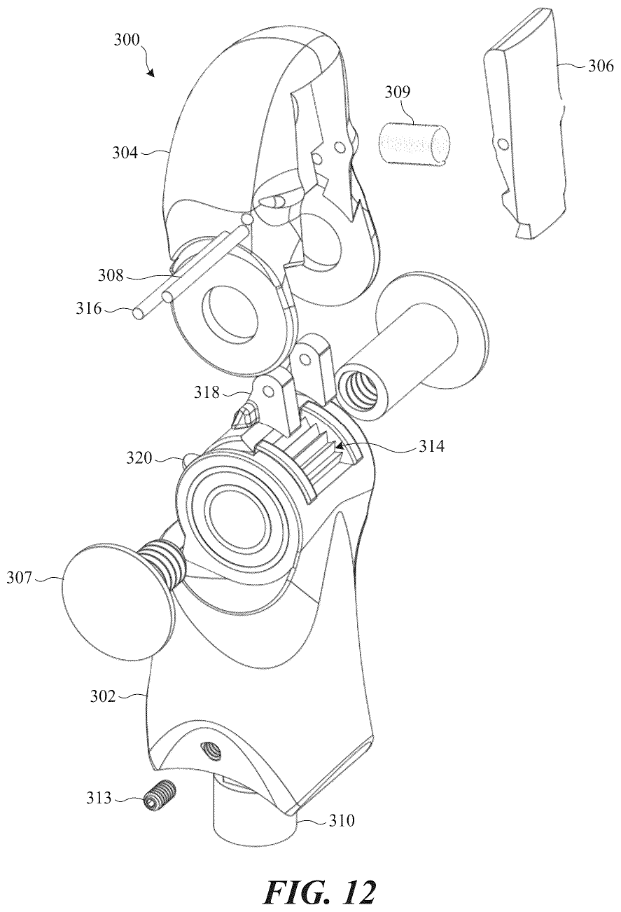

The lever 306 can prevent movement of the second phalange 304 in extension and/or flexion. The lever 306 can be coupled to the second phalange 304 via a pin 308 (shown in FIG. 12). The pin 308 may pivotally couple the lever 306 to the second phalange 304 and allow the lever 306 to move (e.g., rotate, pivot, etc.) between a first position and a second position. In the first position, the lever 306 can prevent movement of the second phalange 304 in extension (e.g., by engaging with a set of teeth 314, shown in FIG. 12), while allowing movement of the second phalange 304 in flexion. For example, the prosthetic finger 300 can be a passive prosthetic finger in which a user moves the second phalange 304 to a desired position by manually causing the second phalange 304 to move in flexion. As the prosthetic finger 300 moves in flexion, the lever 306 is in the first position and prevents movement of the prosthetic finger 300 in extension.

In the second position, the lever 306 can allow movement of the second phalange 304 in extension and flexion (e.g., by disengaging with the set of teeth 314, discussed with respect to FIG. 12). In some implementations, the prosthetic finger 300 includes a first spring element 309 (e.g., a spring, a coil spring, etc.) that biases the lever 306 in the first position. A user input can actuate the lever 306 to the second position, for example, by rotating/pivoting the lever 306 about the pin 308. In the second position, the lever 306 disengages with the set of teeth 314 (shown in FIG. 12) to allow movement in extension.

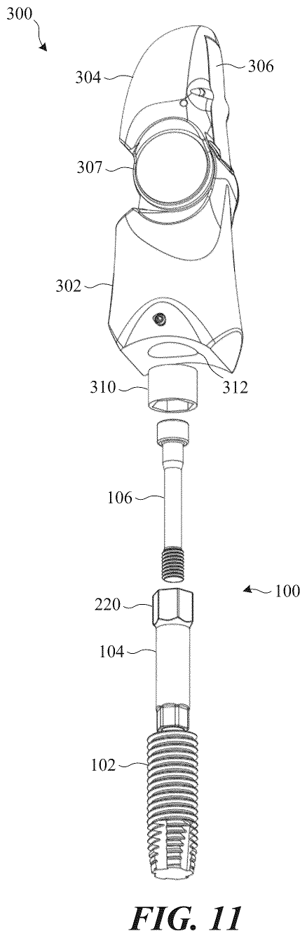

Referring now to FIG. 11, an exploded view of the prosthetic finger 300 is shown. As shown in FIG. 11, the prosthetic finger 300 can include a collar 310 that couples the first phalange 302 to the bone implant 100. The collar 310 can attach to the abutment 104. For example, the abutment 104 can include the head portion 220 which is positioned outside the body of a user when the bone implant 100 is attached. The collar 310 can rigidly attach to the abutment 104 such that there is no movement between the collar 310 and the abutment 104 when they are attached. For example, in some cases, the collar 310 can press-fit onto the head portion 220 and the coupling of the collar 310 with respect to the head portion 220 can prevent movement between the collar 310 and the head portion 220.

In some cases, the collar 310 can define an opening that has geometric features that mate with geometric features of the abutment 104. For example, if the head portion 220 of the abutment 104 is hex-shaped, the collar 310 can define an opening having a hex-shaped configuration that mates with the head portion 220. Additionally or alternatively, the collar can be attached to the abutment using other methods, such as the collar 310 and the head portion 220 including threaded features, adhesive materials, welding, and so on. In some cases, the collar 310 can be configured as a multipart component that clamps to the abutment 104. The clamping of the collar 310 can provide forces that prevent movement between the collar 310 and the abutment 104.

The first phalange 302 can couple to the collar 310 and be configured to rotate and/or translate with respect to the collar 310. For example, the first phalange 302 can define an opening 312 that mates with the collar 310. The collar 310 can be positioned within the opening 312 and the first phalange 302 can rotate and/or translate with respect to the collar 310. The assembly can include a retaining mechanism that selectively prevents movement between the first phalange 302 and the collar 310. For example, the retaining mechanism can include a set screw 313 (shown in FIG. 12) that engages with the first phalange 302 and engages with the collar 310 to prevent movement between the first phalange 302 and the collar 310. Additionally or alternatively, the first phalange 302 and collar 310 can include a set of threads or teeth that engage with each other to prevent rotation. Accordingly, the first phalange 302 can be positioned on the collar 310, attached to the abutment 104, and the alignment of the first phalange 302 (and prosthetic finger 300) can be adjusted with respect to the bone implant 100. Once in a desired position, the retaining mechanism can be engaged to lock the first phalange 302 in place and prevent further movement between the bone implant 100 and the prosthetic finger 300.

FIG. 12 shows an exploded view of the prosthetic finger 300 shown in FIGS. 9 and 10. As shown in FIG. 12, the first phalange 302 can couple to the collar 310, and the collar 310 can attach to the bone implant 100. The second phalange 304 can rotate with respect to the first phalange 302 to move in flexion and extension.

The prosthetic finger 300 can include a second spring element (similar to the second spring element 216), which biases the second phalange 304 to move in extension. For example, when the lever 306 is in the second position the spring element can cause the second phalange 304 to move in extension. When the lever 306 is in the first position, the engagement of the lever 306 with the first phalange 302 (e.g., the set of teeth 314) can prevent movement in extension and the spring element may exert a force on the lever 306 and the first phalange 302.

The prosthetic finger 300 can further include a locking mechanism configured to retain the second phalange 304 at a position of flexion and/or extension. The locking mechanism can include the first phalange 302 and the lever 306. The first phalange 302 can define a set of teeth 314 and the lever 306 can include an engagement feature that engages with the set of teeth 314 (e.g., a ratcheting mechanism) when the lever 306 is in the first position. The set of teeth 314 and the engagement feature can allow movement in a first direction (e.g., flexion) and prevent movement in a second direction (e.g., extension). For example, when the lever 306 is in the first position, the engagement feature can engage with the set of teeth 314 such that movement of the second phalange 304 in flexion is allowed (e.g., the engagement feature slides over the set of teeth 314). With the lever 306 in the first position, attempting to move the second phalange 304 in extension (e.g., by manually moving the second phalange 304) causes the engagement feature to abut against the set of teeth 314 (e.g., one tooth of the set of teeth 314). In this way, when the lever 306 is in the first position, the lever 306 and the first phalange 302 allow movement of the second phalange 304 in flexion while preventing movement of the second phalange 304 in extension. Furthermore, engagement between the set of teeth 314 and the engagement feature can act as a locking mechanism, retaining the second phalange 304 at a desired position of flexion (e.g., partially flexed, fully flexed, etc.). When the lever 306 is rotated about the pin 308 into the second position, the engagement feature can disengage from the set of teeth 314 and the second spring element can bias the second phalange 304 into extension.

In some implementations, the prosthetic finger 300 further includes props 318 (e.g., at least one prop, a pair of props, a prop, etc.), which can be an example of the props described herein (e.g., props 240). The props 318 can be coupled to the second phalange 304 using a pin 316 (e.g., a bar, a shaft, etc.) or another rotational coupling mechanism (e.g., bearings, etc.). The props 318 can facilitate automatic return of the prosthetic finger 300 to an extended configuration (e.g., as shown in FIG. 9) in response to reaching a defined flexion point (e.g., a maximum flexion, a threshold flexion point, etc.). For example, the first phalange 302 can include stops 320 (e.g., at least one stop, a pair of stops, a stop, etc.). The props 318 can be coupled to the second phalange 304 and configured to contact the stops 320 at the defined flexion point. The contact of the props 318 with the stops 320 can cause the props 318 to transition the lever 306 from the first position to the second position, thereby allowing the second spring element to cause the second phalange 304 to move in extension. The props 318 can be configured such that the props 318 do not contact the lever 306 until the defined flexion point is reached, whereby the props 318 can transition the lever 306 to the second position.

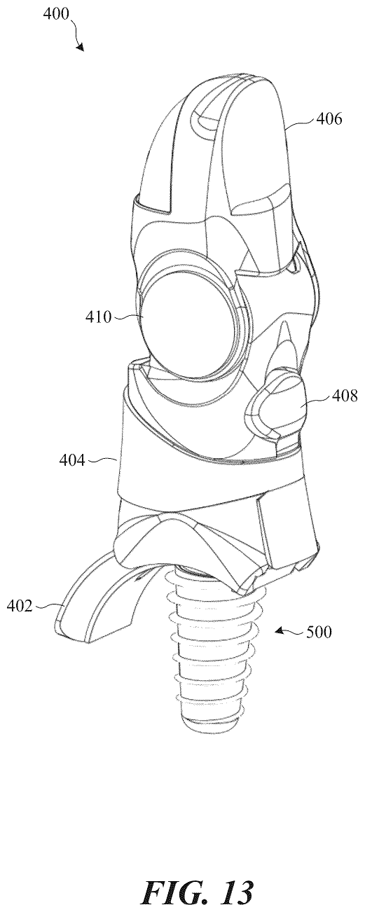

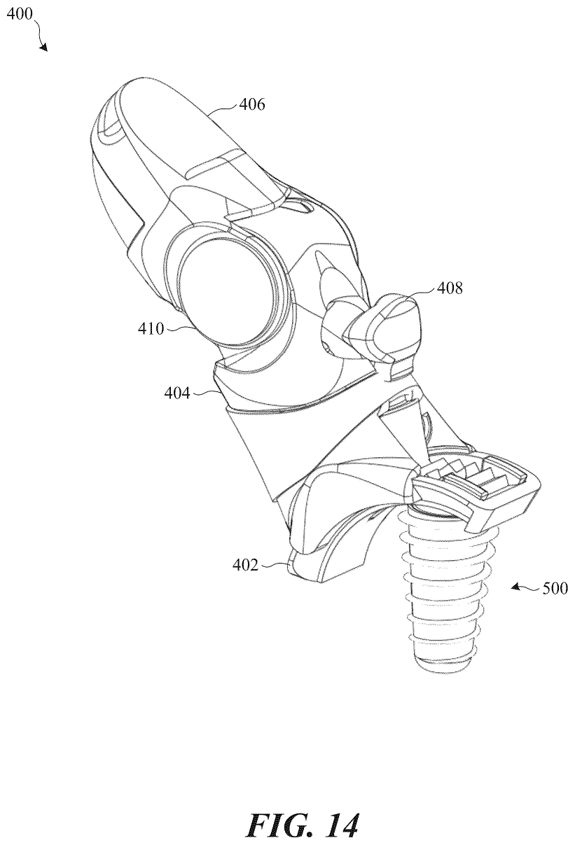

FIGS. 13 and 14 show an example prosthetic finger 400 that attaches to a bone implant 500, as described herein. FIG. 13 shows an example of the prosthetic finger 400 in an extended configuration and FIG. 14 shows an example of the prosthetic finger 400 in a partially flexed configuration.

The prosthetic finger 400 can include a track 402, a first phalange 404, a second phalange 406, and a lever 408. The first phalange 404 can have a first end coupled to the track 402 and be configured to move along the track 402. For example, the first phalange 404 can be coupled to the track 402 and slide along a profile defined by the track 402. The track 402 can allow movement of the first phalange 404 in flexion and extension. The bone implant 500 can be an example of the bone implants described herein and include an anchor 502, an abutment 504, and a connector 506.

In some implementations, the track 402 can have a constant radius of curvature such that the first phalange 404 moves along the track 402 in a circumferential path. For example, a shape of the track 402 can be curved. As the first phalange 404 moves along the curve of the track 402, a center of motion of the first phalange 404 can be located at an axis of curvature of the track 402 (which may be referred to as a “remote center of motion” or “virtual joint center”). In some implementations, the combination of the curvature of the track 402 and an attachment location of the track 402 on the bone implant 500 can position the center of motion (remote center of motion) within the hand or at a position that is remote from the mounting location of the track 402. Such configurations can result in an externally mounted device such as the prosthetic finger 400 being able to move substantially similar to an internal joint such as a natural finger or thumb joint. In this regard, the shape of the track 402 and its orientation on the user can be configured such that the movement of the prosthetic finger 400 can achieve similar motion to the motion of a natural digit.

The second phalange 406 can be coupled to the first phalange 404 and configured to move in flexion and extension relative to the first phalange 404. For example, the prosthetic finger 400 can include one or more rotational structures (e.g., bearings, pins, etc.), shown as pin 410 (e.g., a shaft, a bar, a threaded pin, etc.), that pivotally couples the second phalange 406 to the first phalange 404 and allows the second phalange 406 to rotate with respect to the first phalange 404.

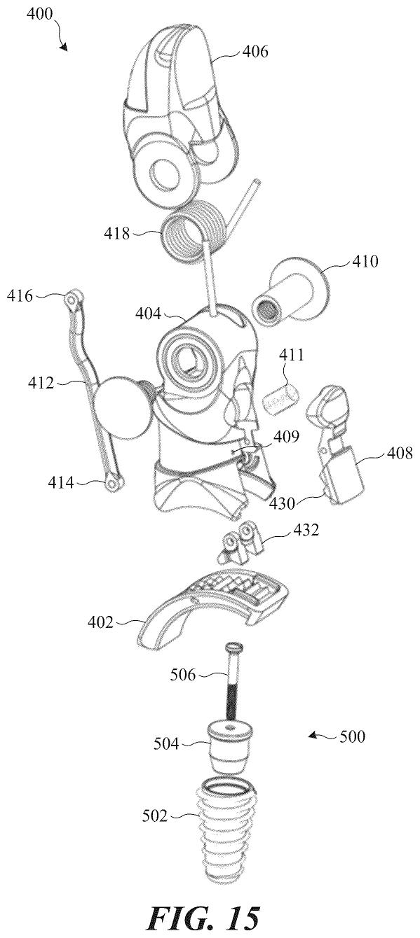

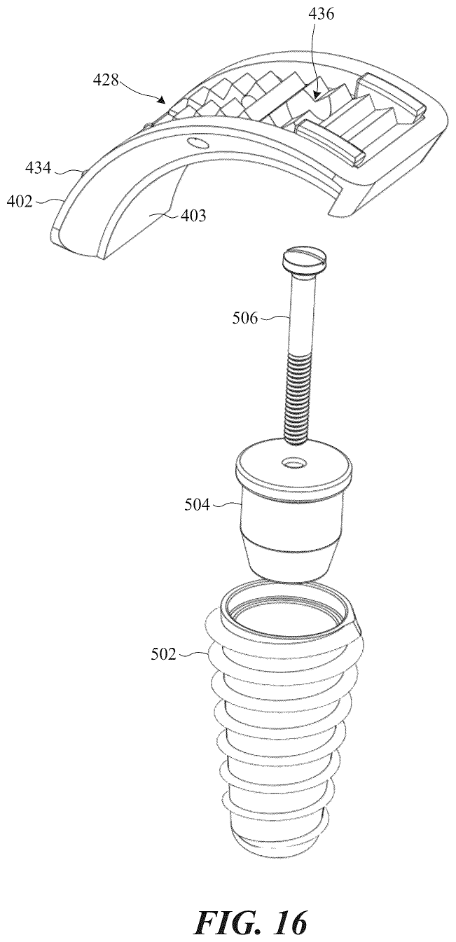

The lever 408 can be an example of the levers described herein (e.g., lever 208 and lever 306) and be configured to prevent movement of the first phalange 404 and/or the second phalange 406 in extension and/or flexion. The lever 408 can be coupled to the first phalange 404 via a pin 409 (shown in FIG. 15). The pin 409 may pivotally couple the lever 408 to the first phalange 404 such that the lever 408 can move (e.g., rotate, pivot, etc.) between a first position and a second position. In the first position the lever 408 can engage with the track 402 (e.g., by engaging with a set of teeth 428, discussed with respect to FIG. 16) to prevent movement of the first phalange 404 and the second phalange 406 in extension while allowing movement of the first phalange 404 and the second phalange 406 in flexion. For example, the prosthetic finger 400 can be a passive prosthetic finger in which a user moves the phalanges to a desired position by manually causing the first phalange 404 and the second phalange 406 to move in flexion. As the prosthetic finger 400 is moved in flexion, the lever 408 is in the first position and engages with the track 402 to prevent movement of the prosthetic finger 400 in extension.

In the second position, the lever 408 can disengage with the track 402 (e.g., disengages with the set of teeth 428) to allow movement of the first phalange 404 and/or the second phalange 406 in extension and flexion (e.g., free movement of the prosthetic finger 400). In some implementations, the prosthetic finger 400 includes a first spring element 411 (e.g., a spring, a coil spring, etc.) that biases the lever 408 in the first position. The first spring element 411 can be coupled to the first phalange 404 and can provide a biasing force that urges the lever 408 into the first position. A user input can actuate the lever 408 to the second position, for example, by rotating/pivoting the lever 408 about the pin 409 to disengage the lever 408 from the track 402 and to allow movement of the first phalange 404 and/or the second phalange 406 in extension.

FIG. 15 shows an exploded view of the prosthetic finger 400 shown in FIGS. 13 and 14. As shown in FIG. 15, the prosthetic finger 400 can include a link bar 412, which can be an example of the link bars (e.g., link bar 218) described herein. The link bar 412 can couple motion of the first phalange 404 to motion of the second phalange 406. For example, the link bar 412 can cause the first phalange 404 and the second phalange 406 to both move in flexion or both move in extension, as described herein.

The link bar 412 can include a first link bar end 414 coupled to the track 402 and a second link bar end 416 coupled to the second phalange 406. The link bar 412 can rotate with respect to the track 402 and the second phalange 406. For example, the first link bar end 414 can rotate with respect to the track 402 and the second link bar end 416 can rotate with respect to the second phalange 406. In some cases, the link bar 412 can be attached to each of the track 402 and the second phalange 406 using pins or any other suitable rotational coupling. As described herein, the link bar 412 can couple motion of the first phalange 404 and the second phalange 406.

The prosthetic finger 400 can further include a second spring element 418 (e.g., a spring, a spring element, a torsional spring, etc.) coupled to the first phalange 404 and/or the second phalange 406. In various implementation, the second spring element 418 is coupled to the pin 410 (e.g., wound about). The second spring element 418 can provide a biasing force that urges the first phalange 404 and the second phalange 406 to move in extension. For example, when the lever 408 is in the second position (e.g., disengaged from the track 402) the second spring element 418 can cause the first phalange 404 and the second phalange 406 to move in extension. When the lever 408 is in the first position, the engagement of the lever 408 with the track 402 (e.g., the set of teeth 428) can prevent movement in extension and the second spring element 418 may exert a force on the lever 408 and the track 402.

Referring now to FIGS. 15 and 16, the prosthetic finger 400 can further include a locking mechanism configured to retain the prosthetic finger 400 at a position of flexion and/or extension. The locking mechanism can include the track 402 and the lever 408. As shown in FIG. 16, the track 402 can define a set of teeth 428 and the lever 408 can include an engagement feature 430 that engages with the set of teeth 428. The set of teeth 428 and the engagement feature 430 can allow movement in a first direction (e.g., flexion) and prevent movement in a second, opposite direction (e.g., extension). For example, when the lever 408 is in the first position, the engagement feature 430 can engage with the set of teeth 428 such that movement of the prosthetic finger 400 in flexion is allowed (e.g., the engagement feature 430 slides over the set of teeth 428). With the lever 408 in the first position, attempting to move the prosthetic finger 400 in extension (e.g., by manually moving the first phalange 404 and/or the second phalange 406) causes the engagement feature 430 to abut against the set of teeth 428 (e.g., one tooth of the set of teeth 428), thereby preventing movement of the prosthetic finger 400 in extension. Furthermore, engagement between the set of teeth 428 and the engagement feature 430 acts as a locking mechanism, retaining the first phalange 404 and the second phalange 406 at a desired position of flexion (e.g., partially flexed, fully flexed, etc.). When the lever 408 is rotated about the pin 409 into the second position, the engagement feature 430 disengages from the set of teeth 428 and the second spring element 418 biases the first phalange 404 and the second phalange 406 into extension.

In some implementations, the prosthetic finger 400 can include props 432 (e.g. at least one prop, a pair of props, etc.), which can be an example of the props described herein (e.g., props 240). The props 432 can facilitate automatic return of the prosthetic finger 400 to an extended configuration (e.g., as shown in FIG. 13) in response to reaching a defined flexion point (e.g., a maximum flexion, a threshold flexion value, etc.). For example, the prosthetic finger 400 can include one or more stops 434 (e.g., at least one stop, a pair of stops, a stop, etc.) coupled to the track 402. The props 432 can be coupled to the first phalange 404 and configured to contact the stops 434 at the defined flexion point. The contact of the props 432 with the stops 434 can cause the props 432 to contact the lever 408 and transition the lever 408 from the first position to the second position. When the lever 408 is in the second position, the second spring element 418 can cause the first phalange 404 and the second phalange 406 to move in extension. The props 432 can be configured such that the props 432 do not contact the lever 408 until the defined flexion point is reached, whereby the props 432 allow the lever 408 to transition to the second position.

As shown in FIG. 16, the track 402 can be coupled to the anchor 502 by the abutment 504 and the connector 506. In some cases, the track 402 can define an inner surface 403 having a concave profile. The inner surface 403 may be positioned against a user when the track 402 is coupled to the bone implant 500. In some cases, the inner surface 403 can include cushioning, and/or other surface features that facilitate placement of the track 402 on a skin surface of a person. In some cases, dressing or other materials can be positioned between the track 402 and a skin surface, for example to facilitate healing of the skin, reduce potential for infection, etc.

The anchor 502 may be attached to a bone and positioned in the body under a skin surface of a person as described herein. The abutment 504 may interface with the anchor and function as an adapter between the anchor 502 and the connector 506. For example, the abutment 504 may include threaded features which rigidly fix the abutment 504 to the anchor 502 and prevents movement between the abutment 504 and the anchor 502. Additionally or alternatively, the abutment 504 may be adhesively coupled to the anchor 502 or fixed to the anchor 502 using any other suitable methods. The abutment 504 can include internal threaded features and the connector 506 can couple to the abutment 504 using the internal threaded features. In other implementations, the connector 506 can couple to the abutment 504 using any suitable method.

In some implementations, the abutment 504 may be contained within the body of the user, for example completely positioned under the skin surface of the user. In other implementations, a portion of the abutment 504 may extend through the skin surface of the user. In such implementations, having a portion of the abutment 504 extend through the skin surface may facilitate attachment and removal of the track 402 without disrupting the interface between the skin and the abutment 504.

The track 402 can include one or more features that couple the track 402 to the bone implant 500. For example, the track can define an opening 436. The connector 506 can engage with the track 402 and extend through the opening 436 to couple the track 402 to the abutment 504.

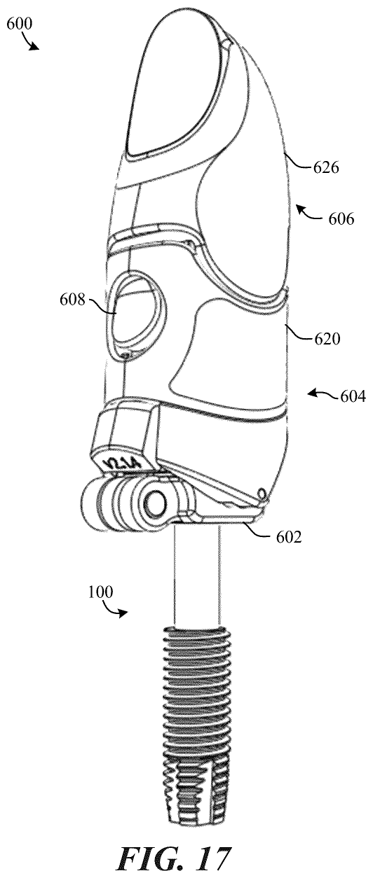

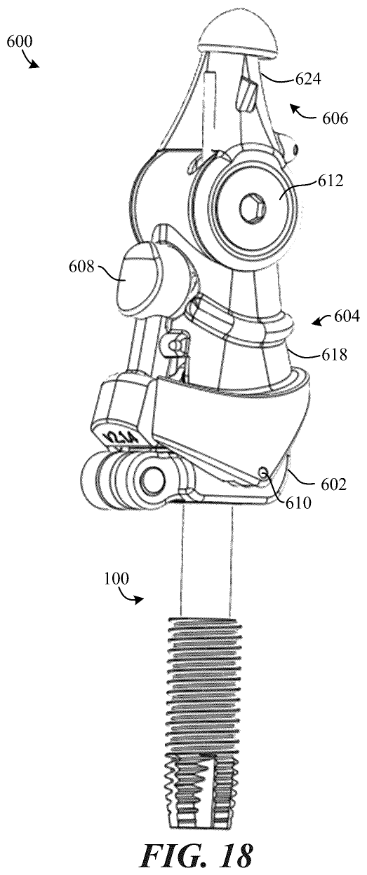

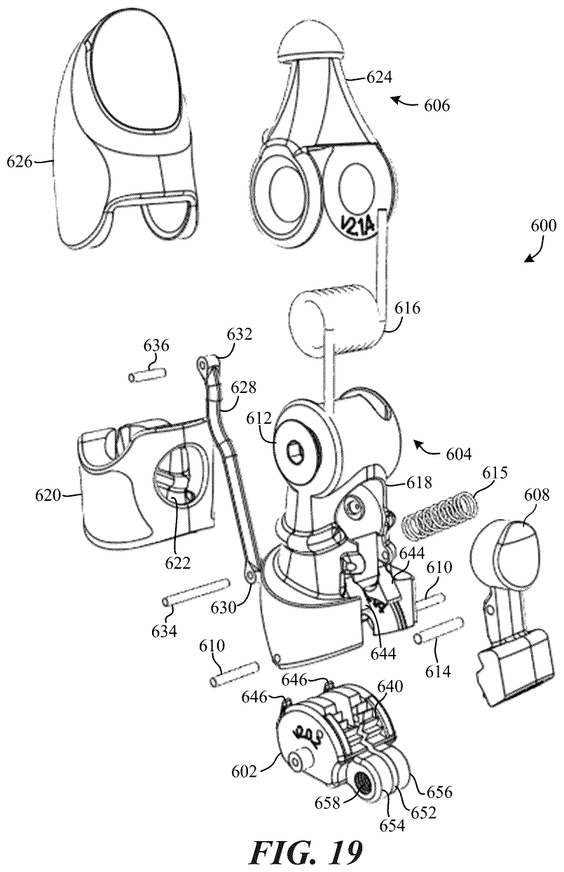

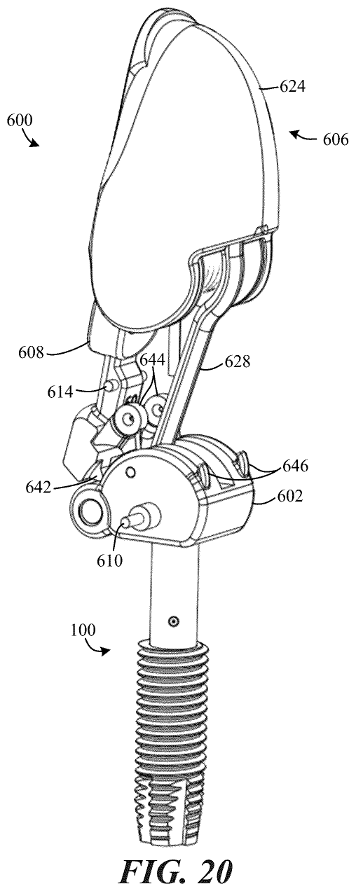

Referring now to FIGS. 17-20, an example prosthetic finger 600 that attaches to the bone implant 100 is shown. FIG. 17 shows an example of the prosthetic finger 600 in an extended and assembled configuration. FIG. 18 shows a partial view of the prosthetic finger 600. FIG. 19 shows an exploded view of the prosthetic finger 600 and FIG. 20 shows another partial view of the prosthetic finger 600. The prosthetic finger 600 can incorporate features of the prosthetic finger 200 as described with respect to FIGS. 3-8.

The prosthetic finger 600 can include a hub 602, a first phalange 604, a second phalange 606, and lever 608. The hub 602 can be substantially similar to the hub 202, the first phalange 604 can be substantially similar to the first phalange 204, and the second phalange 606 can be substantially similar to the second phalange 206. As described in more detail herein, the hub 602 is configured to be coupled to the bone implant 100 (e.g., at the abutment 104). The first phalange 604 can have a first end coupled to the hub 602 and can move in flexion and extension relative to the hub 602. For example, the prosthetic finger 600 can include one or more rotational structures (e.g., bearings, pins, etc.), shown as pin(s) 610 (e.g., shafts, bars, etc.) that pivotally couples the first phalange 604 to the hub 602 and allows the first phalange 604 to rotate with respect to the hub 602. The second phalange 606 can be coupled to the first phalange 604 and configured to move in flexion and extension relative to the first phalange 604 and/or the hub 602. For example, the prosthetic finger 600 can include one or more rotational structures (e.g., bearings, pins, etc.), shown as pin(s) 612 (e.g., shafts, bars, threaded pins, etc.), that pivotally couples the second phalange 606 to the first phalange 604 and allows the second phalange 606 to rotate with respect to the first phalange 604.

The lever 608 can prevent movement of the first phalange 604 and/or the second phalange 606 in extension and/or flexion, The lever 608 can be coupled to the first phalange 604 via a pin 614 (e.g., a shaft, a bar, etc.). The pin 614 may pivotally couple the lever 608 to the first phalange 604 such that the lever 608 can move (e.g., rotate, pivot, etc.) between a first position and a second position. In the first position, the lever 608 can engage with the hub 602 (e.g., by engaging with a set of teeth 640, discussed with respect to FIG. 15) to prevent movement of the first phalange 604 and the second phalange 606 in extension while allowing movement of the first phalange 604 and the second phalange 606 in flexion. For example, the prosthetic finger 600 can be a passive prosthetic finger in which the user moves the first phalange 604 and/or the second phalange 606 to a desired position by manually causing the first phalange 604 and/or the second phalange 606 to move in flexion. As the prosthetic finger 600 is moved in flexion, the lever 608 is in the first position and engages with the hub 602 to prevent movement of the prosthetic finger 600 in extension.

In the second position, the lever 608 can disengage with the hub 602 (e.g., disengages with the set of teeth 640, discussed with respect to FIG. 15) to allow movement of the first phalange 604 and/or the second phalange 606 in extension and flexion (e.g., free movement of the prosthetic finger 600). In some implementations, the prosthetic finger 600 includes a first spring element 615 (e.g., a spring, a coil spring, etc.) that biases the lever 608 in the first position. The first spring element 615 can be coupled to the first phalange 604 and can provide a biasing force that urges the lever 608 into the first position. A user input can actuate the lever 608 to the second position, for example, by rotating/pivoting the lever 608 about the pin 614 to disengage the lever 608 from the hub 602 and allow movement of the first phalange 604 and/or the second phalange 606 in extension and flexion.

As shown in FIG. 19, the prosthetic finger 600 can further include a second spring element 616 (e.g., a spring, a spring element, a torsional spring, etc.) coupled to the first phalange 604 and/or the second phalange 606. In various implementations, the second spring element 616 is coupled to the pin(s) 612 (e.g., wound about). The second spring element 616 can provide a biasing force that urges the first phalange 604 and the second phalange 606 to move in extension. For example, when the lever 608 is in the second position (e.g., disengaged from the hub 602) the second spring element 616 can cause the first phalange 604 and the second phalange 606 to move in extension. When the lever 608 is in the first position, the engagement of the lever 608 with the hub 602 (e.g., the set of teeth 640) can prevent movement in extension and the second spring element 616 may exert a force on the lever 608 and the hub 602.

As shown in FIGS. 17-19, the first phalange 604 can include a first phalange frame 618 (e.g., a structure, a support) and a first phalange cover 620 (e.g., a housing, an external appearance, etc.). The first phalange frame 618 can be coupled to the hub 602 (e.g., via the pin(s) 610). The first phalange cover 620 can be coupled to the first phalange frame 618. For example, the first phalange frame 618 can include one or more protrusions (e.g., flanges, snap-fit connections, etc.) configured to engage with one or more recesses (e.g., apertures, snap-fit connections, etc.) of the first phalange cover 620. As show in FIGS. 17 and 19, the first phalange cover 620 includes an aperture 622 (e.g., an opening, etc.) configured to allow for movement and/or actuation of the lever 608 between the first position and the second position,

Similarly, the second phalange 606 can include a second phalange frame 624 (e.g., a structure, a support, etc.) and a second phalange cover 626 (e.g., a housing, an external appearance, etc.). The second phalange frame 624 can be coupled to the first phalange frame 618 (e.g., via the pin(s) 612). The second phalange cover 626 can be coupled to the second phalange frame 624. For example, the second phalange frame 624 can include one or more protrusions (e.g., flanges, snap-fit connections, etc.) configured to engage with one or more recesses (e.g., apertures, snap-fit connections, etc.) of the second phalange cover 626.

In various implementations, the first phalange cover 620 and/or the second phalange cover 626 are configured to make the prosthetic finger 600 appear similar to a human finger. For example, the second phalange cover 626 can include one or more structures resembling a human fingernail.

Referring now to FIGS. 19 and 20, the prosthetic finger 600 can further include a link bar 628 that couples motion of the first phalange 604 to motion of the second phalange 606. The link bar 628 can be substantially similar to the link bar 218. For example, the link bar 628 can cause the first phalange 604 and the second phalange 606 to both move in flexion or both move in extension. Accordingly, if a user manually moves the second phalange 606 in flexion, the link bar 628 can cause the first phalange 604 to also move in flexion. If a user manually moves the first phalange 604 in flexion, the link bar 628 can cause the second phalange 606 to also move in flexion. Accordingly, the link bar 628 can couple the movements of the first phalange 604 and the second phalange 606.

As shown in FIG. 19, the link bar 628 can include a first link bar end 630 coupled to the hub 602 and a second link bar end 632 coupled to the second phalange 606. The link bar 628 can rotate with respect to the hub 602 and the second phalange 606. For example, the first link bar end 630 can rotate about the hub 602 and the second link bar end 632 can rotate about the second phalange 606. The first link bar end 630 can be pivotally coupled to the hub 602 via a pin 634 and the second link bar end 632 can be pivotally coupled to the second phalange 606 via a pin 636 (e.g., a shaft, a bar, etc.).

As shown in FIGS. 19 and 20, the prosthetic finger 600 can further include a locking mechanism configured to retain the prosthetic finger 600 at a position of flexion and/or extension. The locking mechanism can include the hub 602 and the lever 608. For example, the hub 602 can define a set of teeth 640 and the lever 608 can include an engagement feature 642 that engages with the set of teeth 640 (e.g., a ratcheting mechanism) when the lever 608 is in the first position. The set of teeth 640 and the engagement feature 642 can allow movement in a first direction (e.g., flexion) and prevent movement in a second, opposite direction (e.g., extension). For example, when the lever 608 is in the first position, the engagement feature 642 can engage with the set of teeth 640 such that movement of the prosthetic finger 600 in flexion is allowed (e.g., the engagement feature 642 slides over the set of teeth 640). With the lever 608 in the first position, attempting to move the prosthetic finger 600 in extension (e.g., by manually moving the first phalange 604 and/or the second phalange 606) causes the engagement feature 642 to abut against the set of teeth 640 (e.g., one tooth of the set of teeth 640), thereby preventing movement of the prosthetic finger 600 in extension. In this way, when the lever 608 is in the first position, the lever 608 and the hub 602 allow movement of the prosthetic finger 600 in flexion while preventing movement of the prosthetic finger 600 in extension. Furthermore, engagement between the set of teeth 640 and the engagement feature 642 acts as a locking mechanism, retaining the first phalange 604 and the second phalange 606 at a desired position of flexion (e.g., partially flexed, fully flexed, etc.). When the lever 608 is rotated about the pin 614 into the second position, the engagement feature 642 disengages from the set of teeth 640 and the second spring element 616 biases the first phalange 204 and the second phalange 206 into extension.

In some implementations, and shown in FIG. 20, the prosthetic finger 600 further includes props 644 (e.g., at least one prop, a pair of props, a prop, etc.). The props 644 can facilitate automatic return of the prosthetic finger 600 to an extended configuration in response to reaching a defined flexion point (e.g., maximum flexion, a threshold flexion value, etc.). For example, the prosthetic finger 600 can further include stops 646 (e.g., at least one stop, a pair of stops, a stop, etc.) coupled to the hub 602. The props 644 can be coupled to the first phalange 604 and configured to contact the stops 646 at the defined flexion point. The contact of the props 644 with the stops 646 can cause the props 644 to contact the lever 608 and transition the lever 608 from the first position to the second position. When the lever 608 is in the second position, the second spring element 616 can cause the first phalange 604 and the second phalange 606 to move in extension. The props 644 can be configured such that the props 644 do not contact the lever 608 until the defined flexion point is reached, whereby the props 644 transition the lever 608 to the second position.

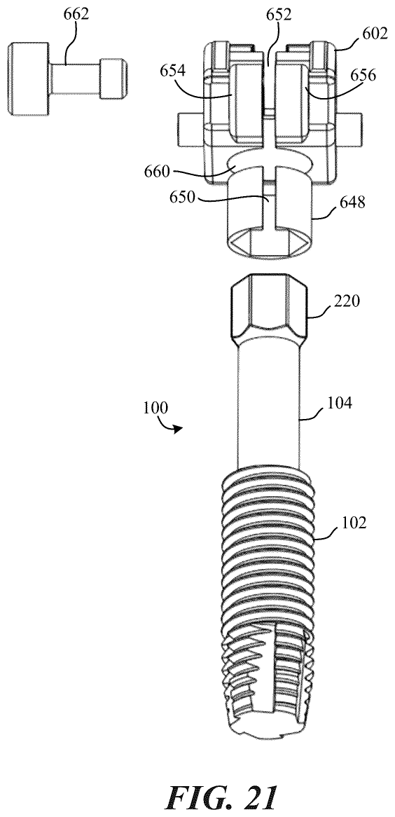

Referring now to FIG. 21, the prosthetic finger 600 can further include a retaining mechanism for fixing the hub 602 to the bone implant 100. As shown in FIG. 21, the prosthetic finger 600 includes a collar 648. The collar 648 can attach to the abutment 104. For example, the head portion 220 of the abutment 104 can be positioned outside the body of the user when the bone implant 100 is attached to the bone. The collar 648 can rigidly attach to the abutment 104 such that there is no movement between the collar 648 and the abutment 104.

In some implementations, the collar 648 defines an opening that has geometric features that mate with geometric features of the abutment 104. For example, if the head portion 220 of the abutment 104 is hex-shaped, the collar 648 can define an opening having a hex-shaped configuration that mates with the head portion 220.

As shown in FIG. 21, the collar 648 includes a slot 650 (e.g., an aperture, a separation, a slit etc.) extending along a longitudinal axis (e.g., a length axis) of the collar 648. As shown in FIG. 19, the hub 602 includes a slot 652, a first protrusion 654 (e.g., a tab), and a second protrusion 656 (e.g., a tab). The first protrusion 654 can extend (e.g., extend outward) from an outer surface of the hub 602 and can be positioned on a first side of the slot 652. The second protrusion 656 can extend (e.g., extend outward) from an outer surface of the hub 602 and can be positioned on a second side of the slot 652. In this way, the first protrusion 654 and the second protrusion 656 are positioned a distance apart as defined by a width of the slot 652. The first protrusion 654 can define an aperture 658 (e.g., a hole, an opening, etc.) extending through a width of the first protrusion 654. Similarly, the second protrusion 656 can define an aperture (not shown) extending through a width of the second protrusion 656. The aperture 658 of the first protrusion 654 can be aligned with the aperture of the second protrusion 656 such that the two apertures share a common central axis.

As shown in FIG. 21, the hub 602 can further include an opening 660 (e.g., an opening, a hole, etc.) configured to mate with the collar 648. The collar 648 can be positioned within the opening 660 and the slot 650 of the collar 648 can be aligned with the slot 652 of the hub 602. A screw 662 can extend through the aperture 658 of the first protrusion 654 and the aperture of the second protrusion 656. Tightening of the screw 662 can draw the first protrusion 654 and the second protrusion 656 toward one another, thereby acting as a clamp that reduces a width of the slot 652. Tightening of the screw 662 can also reduce a width of the slot 650. In this way, tightening of the screw 662 can clamp the collar 648 onto the head portion 220 of the abutment 104 and simultaneously clamp the hub 602 onto the collar 648. In this way, the retaining mechanism of the prosthetic finger 600 includes the hub 602, the collar 648, and the screw 662 and facilitates secure fastening of the hub 602 to the abutment 104.

In some implementations, the prosthetic finger 600 is configured for use as a thumb. In other implementations, the prosthetic finger 600 is configured for use as a partial digit (e.g., attached at a proximal interphalangeal joint). A size and/or shape of the first phalange 604 and/or the second phalange 606 may be selected based at least on an intended use of the prosthetic finger 600 (e.g., use as a thumb, use as a partial digit). Furthermore, a positioning of the prosthetic finger 600 relative to the bone implant 100 may be selected based on the intended use of the prosthetic finger 600 (e.g., oriented at an angle for a thumb, aligned with the proximal phalanges for a partial digit, etc.).

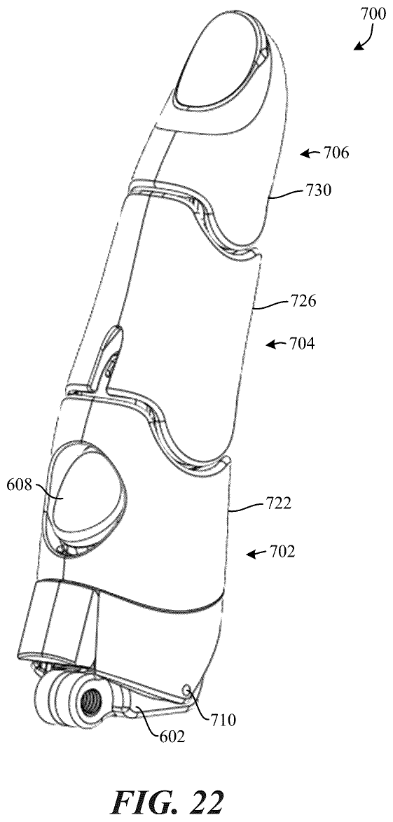

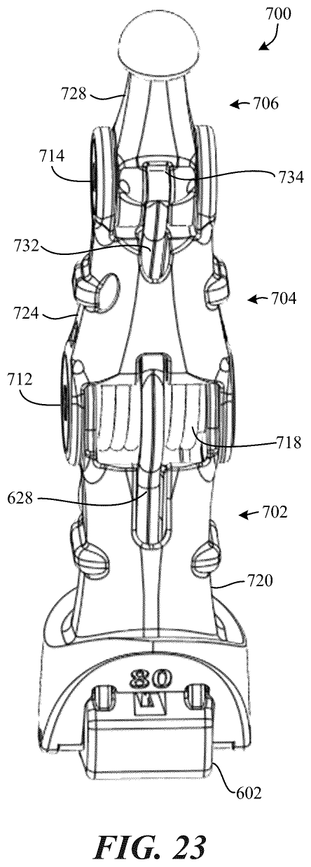

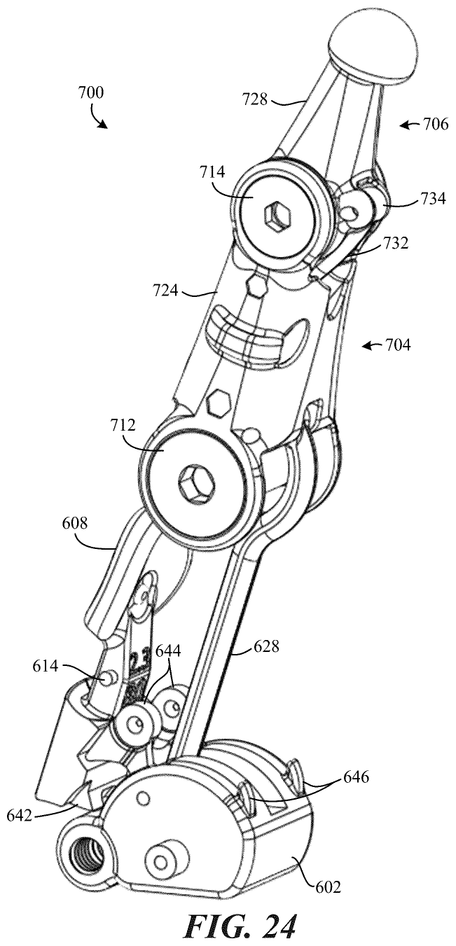

Referring now to FIGS. 22-24, an example prosthetic finger 700 that attaches to the bone implant 100 is shown. FIG. 22 shows an example of the prosthetic finger 700 in an extended and assembled configuration. FIG. 23 shows a partial view of the prosthetic finger 700 and FIG. 24 shows another partial view of the prosthetic finger 700. The prosthetic finger 700 can operate in a manner that is substantially similar to the operation of the prosthetic finger 200 and/or the operation of the prosthetic finger 600 (e.g., the same except for an additional phalange), such as the coupling between phalanges and the movement between an extended configuration and a flexed configuration. Furthermore, the prosthetic finger 700 can incorporate features of the prosthetic finger 200, as described with respect to FIGS. 3-8, and features of the prosthetic finger 600, as described with respect to FIGS. 17-20.

The prosthetic finger 700 is configured to be coupled to the hub 602 and the hub 602 is configured to be coupled to the bone implant 100. As shown in FIG. 22, the prosthetic finger 700 can include a first phalange 702, a second phalange 704, a third phalange 706, and the lever 608. The first phalange 702 can be substantially similar to the first phalange 204 and/or the first phalange 604. The first phalange 702 can have a first end coupled to the hub 602 and can move in flexion and extension relative to the hub 602. For example, the prosthetic finger 700 can include one or more rotational structures (e.g., bearings, pins, etc.), shown as pin(s) 710, that pivotally couples the first phalange 702 to the hub 602 and allows the first phalange 702 to rotate with respect to the hub 602. The second phalange 704 can be coupled to the first phalange 702 and configured to move in flexion and extension relative to the first phalange 702 and/or the hub 602. For example, the prosthetic finger 700 can include one or more rotational structures (e.g., bearings, pins, etc.), shown as pin(s) 712 (e.g., shafts, bars, threaded pins, etc.), that pivotally couples the second phalange 704 to the first phalange 702 and allows the second phalange 704 to rotate with respect to the first phalange 702. The third phalange 706 can be coupled to the second phalange 704 and configured to move in flexion and extension relative to the second phalange 704. For example, the prosthetic finger 700 can include one or more rotational structures (e.g., bearings, pins, etc.), shown as pin(s) 714, that pivotally couples the third phalange 706 to the second phalange 704 and allows the third phalange 706 to rotate with respect to the second phalange 704.

The lever 608 can prevent movement of the first phalange 702, the second phalange 704, and/or the third phalange 706 in extension and/or flexion by moving between the first position and the second position, as described herein. In the first position, the lever 608 can engage with the hub 602 (e.g., by engaging with the set of teeth 640) to prevent movement of the first phalange 702, the second phalange 704, and the third phalange 706 in extension while allowing movement of the first phalange 702, the second phalange 704, and the third phalange 706 in flexion. In the second position, the lever 608 can disengage with the hub 602 (e.g., disengages with the set of teeth 640) to allow movement of the first phalange 702, the second phalange 704, and/or the third phalange 706 in extension and flexion (e.g., free movement of the prosthetic finger 700).

As shown in FIG. 23, the prosthetic finger 700 can further include a second spring element 718 (e.g., a spring, a spring element, a torsional spring, etc.) coupled to the first phalange 604 and/or the second phalange 606. In various implementations, the second spring element 718 is coupled to the pin(s) 712 (e.g., wound about). The second spring element 718 can provide a biasing force that urges the first phalange 702, the second phalange 704, and the third phalange 706 to move in extension. For example, when the lever 608 is in the second position (e.g., disengaged from the hub 602) the second spring element 718 can cause the first phalange 702, the second phalange 704, and the third phalange 706 to move in extension. When the lever 608 is in the first position, the engagement of the lever 608 with the hub 602 (e.g., the set of teeth 640) can prevent movement in extension and the second spring element 718 may exert a force on the lever 608 and the hub 602.

As shown in FIGS. 22 and 23, the first phalange 702 can include a first phalange frame 720 (e.g., a structure, a support) and a first phalange cover 722 (e.g., a housing, an external appearance, etc.). The first phalange frame 720 can be coupled to the hub 602 (e.g., via the pin(s) 710). The first phalange cover 722 can be coupled to the first phalange frame 720. For example, the first phalange frame 720 can include one or more protrusions (e.g., flanges, snap-fit connections, etc.) configured to engage with one or more recesses (e.g., apertures, snap-fit connections, etc.) of the first phalange cover 722. The first phalange cover 722 can include an aperture (e.g., an opening, etc.) configured to allow for movement and/or actuation of the lever 608 between the first position and the second position.

Similarly, the second phalange 704 can include a second phalange frame 724 (e.g., a structure, a support, etc.) and a second phalange cover 726 (e.g., a housing, an external appearance, etc.). The second phalange frame 724 can be coupled to the first phalange frame 720 (e.g., via the pin(s) 712). The second phalange cover 726 can be coupled to the second phalange frame 724. For example, the second phalange frame 724 can include one or more protrusions (e.g., flanges, snap-fit connections, etc.) configured to engage with one or more recesses (e.g., apertures, snap-fit connections, etc.) of the second phalange cover 726.

Similarly, the third phalange 706 can include a third phalange frame 728 (e.g., a structure, a support, etc.) and a third phalange cover 730 (e.g., a housing, an external appearance, etc.). The third phalange frame 728 can be coupled to the second phalange frame 724 (e.g., via the pin(s) 714). The third phalange cover 730 can be coupled to the third phalange frame 728. For example, the third phalange frame 728 can include one or more protrusions (e.g., flanges, snap-fit connections, etc.) configured to engage with one or more recesses (e.g., apertures, snap-fit connections, etc.) of the third phalange cover 730.

In various implementations, the first phalange cover 722, the second phalange cover 726, and/or the third phalange cover 730 are configured to make the prosthetic finger 700 appear similar to a human finger. For example, the third phalange cover 730 can include one or more structures resembling a human fingernail.

Referring now to FIGS. 23 and 24, the prosthetic finger 700 can include the link bar 628 to couple motion of the first phalange 702 to motion of the second phalange 704, as described herein. The prosthetic finger 700 can further include a second link bar 732 that couples motion of the third phalange 706 to motion of the first phalange 702. Accordingly, if a user manually moves the third phalange 706 in flexion, the second link bar 732 causes the first phalange 702 to also move in flexion and the link bar 628 causes the second phalange 704 to also move in flexion.

The second link bar 732 can include a first end (not shown) coupled to the first phalange 702 and a second end 734 coupled to the third phalange 706. The second link bar 732 can rotate with respect to the first phalange 702 and the third phalange 706. For example, the first end of the second link bar 732 can be pivotally coupled to the first phalange 702 (e.g., via a pin) and configured to rotate relative to the first phalange 702. Similarly, the second end 734 of the second link bar 732 can be pivotally coupled to the third phalange 706 (e.g., via a pin) and configured to rotate relative to the third phalange 706.

In some implementations, the second link bar 732 is configured to couple motion of the third phalange 706 to motion of the second phalange 704. For example, the first end of the second link bar 732 can be pivotally coupled to the second phalange 704 and the second end 734 can be pivotally coupled to the third phalange 706.

The prosthetic finger 700 can further include the locking mechanism that is configured to retain the prosthetic finger 700 at a position of flexion and/or extension, as described herein. As described with respect to FIGS. 19 and 20, engagement between the set of teeth 640 and the engagement feature 642 acts as a locking mechanism, retaining the first phalange 702, the second phalange 704, and the third phalange 706 at a desired position of flexion (e.g., partially flexed, fully flexed, etc.). The prosthetic finger 700 can also include the props 644 and the stops 646, as described herein.

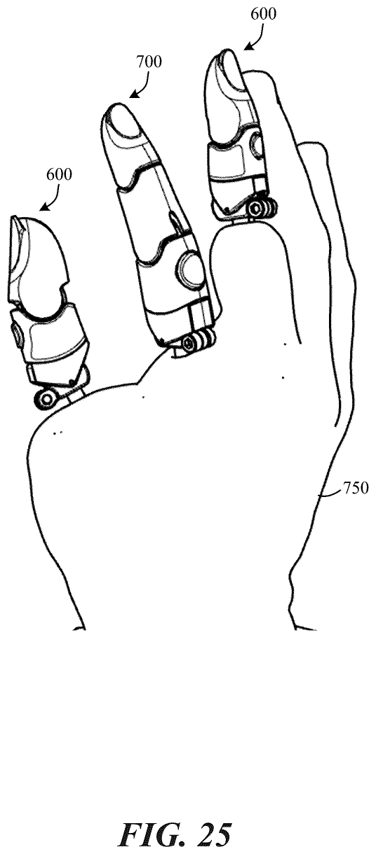

Referring now to FIG. 25, a hand 750 including the prosthetic finger 600 and the prosthetic finger 700 is shown. As shown in FIG. 25, the prosthetic finger 600 can be implemented as a thumb and/or a partial digit. The prosthetic finger 700 can be implemented as a full digit (e.g., an index finger, a middle finger, a ring finger, a pinky finger).

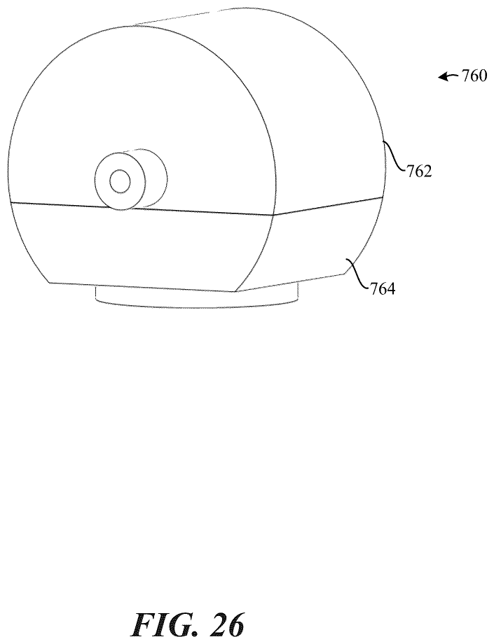

Referring now to FIGS. 26-28, an alternative implementation of a hub 760 is shown. The hub 760 may be coupled to the prosthetic finger 200, the prosthetic finger 600, and/or the prosthetic finger 700 (e.g., in place of the hub 202 and/or the hub 602). As shown in FIG. 26, the hub 760 includes an upper hub 762 and a lower hub 764. The upper hub 762 can define a set of teeth (e.g., the set of teeth 230, the set of teeth 640) and can include stops (e.g., stops 242, stops 646). The upper hub 762 is configured to be removably coupled to the lower hub 764 (e.g., for replacement of the upper hub 762 due to wear of the set of teeth). The lower hub 764 can be coupled to the bone implant 100 (e.g., via the collar 217, via the collar 643, etc.).

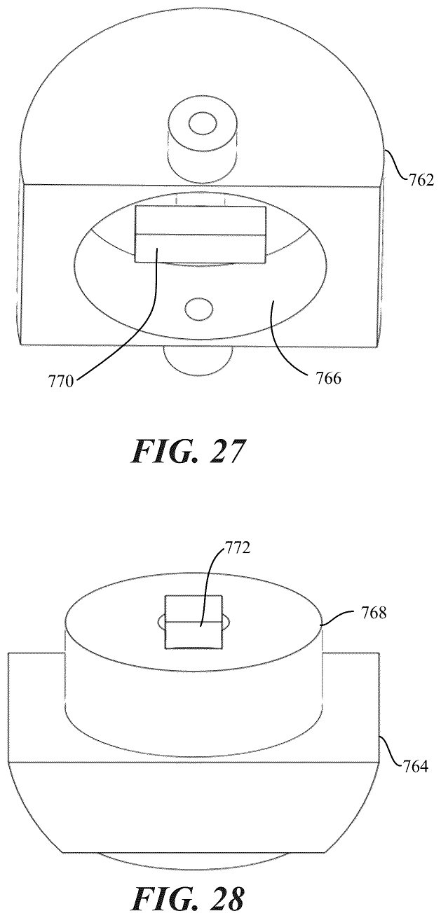

As shown in FIGS. 27 and 28, the upper hub 762 includes an aperture 766 (e.g., an opening or recessed portion), and the lower hub 764 includes a cylindrical protrusion 768. The cylindrical protrusion 768 is configured to be received in the aperture 766 when the upper hub 762 is coupled to the lower hub 764. The upper hub 762 further includes a key 770 (e.g., a rectangular key) extending from an inner surface of the upper hub 762 and through the aperture 766. The lower hub 764 includes a keyway 772 (e.g., an aperture, a rectangular opening, etc.) formed in the cylindrical protrusion 768.

To couple the upper hub 762 to the lower hub 764, the cylindrical protrusion 768 is inserted into the aperture 766 with the key 770 aligned with the keyway 772. The upper hub 762 is then rotated relative to the lower hub 764 (e.g., approximately 90 degrees) so that the key 770 becomes misaligned with the keyway 772, thereby mechanically coupling the upper hub 762 to the lower hub 764. In various implementations, the key 770 and the keyway 772 have rectangular shapes. In other implementations, the key 770 and the keyway 772 can have any other complementary shapes that facilitate coupling between the upper hub 762 and the lower hub 764. In this way, the hub 760 can be assembled without additional mechanical fasteners (e.g., screws), which are often prone to failure.

In some implementations, the key 770 and the keyway 772 are omitted. In such implementations, the aperture 766 can include threads that mesh with threads of the cylindrical protrusion 768.

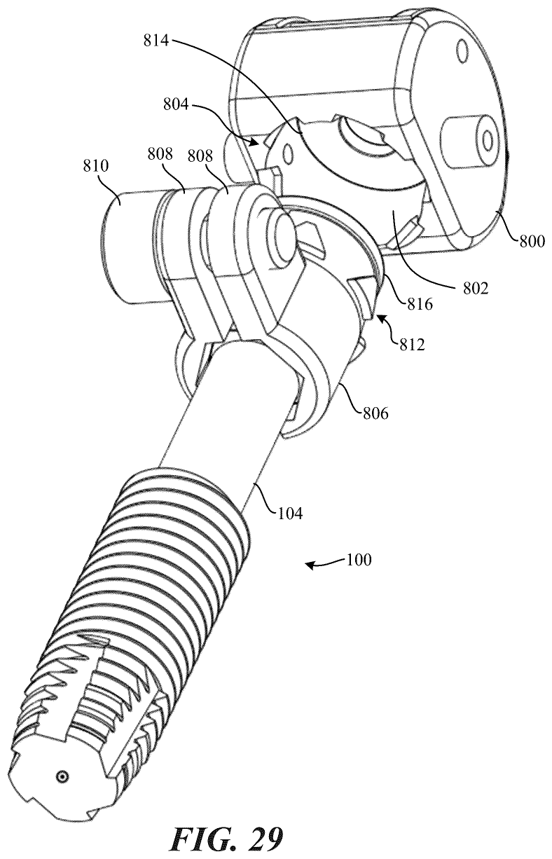

Referring now to FIG. 29, an alternative implementation of a retaining mechanism for coupling a hub 800 to the bone implant 100 is shown. The hub 800 can include features of the hub 202 and/or the hub 602 (e.g., the set of teeth 230, the set of teeth 640, etc.). The hub 800 is configured to be coupled to a prosthetic finger as described herein (e.g., the first phalange 204, the first phalange 604, the first phalange 702, etc.).

As shown in FIG. 29, the hub 800 includes an aperture 802 extending through a bottom surface of the hub 800. The aperture 802 defines a set of teeth 804 (e.g., indents, recesses, etc.) extending along a circumference (e.g., a perimeter, an outer edge, etc.) of the aperture 802. As shown, the bone implant 100 further includes a collar assembly 806. The collar assembly 806 can be coupled to the abutment 104. The collar assembly 806 includes a pair of protrusions 808 extending outward from the collar assembly 806. The protrusions 808 are spaced apart from one another and each define an aperture. A screw 810 extends through the apertures of the protrusions 808. The screw 810 can be tightened to clamp the collar assembly 806 to the abutment 104.

The collar assembly 806 further includes a set of teeth 812 (e.g., protrusions, ridges, etc.) disposed between the protrusions 808 and a top end of the collar assembly 806. The set of teeth 812 extend along a circumference (e.g., a perimeter, an outer edge, etc.) of the collar assembly 806 and are configured to be coupled (e.g., mate, mesh, etc.) to the set of teeth 804 of the hub 800.

The hub 800 further includes a magnet 814 disposed within the aperture 802 (e.g., above the set of teeth 804) and the collar assembly 806 further includes a magnet 816 coupled to the top end of the collar assembly (e.g., above the set of teeth 812). The magnet 814 of the hub 800 is configured to interact (e.g., magnetically attract) with the magnet 816 of the collar assembly 806 to magnetically couple the hub 800 to the bone implant 100.

In various implementations, the coupling between the set of teeth 804 of the hub 800 and the set of teeth 812 of the collar assembly 806 is configured to provide a tunable torque-limiting feature. For example, the set of teeth 804 and the set of teeth 812 can be shaped such that, when a torque applied to the prosthetic finger exceeds a threshold value, the set of teeth 804, 812 generate an axial force that lifts the hub 800 away from the collar assembly 806. This axial displacement can overcome the magnetic coupling between the magnet 814 and the magnet 816, causing the hub 800 (and the attached prosthetic finger) to decouple from the bone implant 100. In this way, the set of teeth 804, 812 and the magnet 814, 816 cooperate to secure the prosthetic finger to the bone implant 100 while also allowing the prosthetic finger to detach from the bone implant 100 when subjected to excessive torque, thereby providing overload protection.

As utilized herein with respect to numerical ranges, the terms “approximately,” “about,” “substantially,” and similar terms generally mean +/−10% of the disclosed values. When the terms “approximately,” “about,” “substantially,” and similar terms are applied to a structural feature (e.g., to describe its shape, size, orientation, direction, etc.), these terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various implementations, are intended to indicate that such implementations are possible examples, representations, or illustrations of possible implementations (and such terms are not intended to connote that such implementations are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain implementations require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary implementations, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above.

It is important to note that the construction and arrangement of the systems, apparatuses, and methods shown in the various example implementations is illustrative only. Additionally, any element disclosed in one implementation may be incorporated or utilized with any other implementation disclosed herein. For example, any of the example implementations described in FIGS. 3-8 of this application can be incorporated with any of the example implementations described in FIGS. 17-24.

What is claimed is:

1. A prosthetic finger comprising:
a hub that attaches to a bone implant;
a first phalange coupled to the hub, wherein the first phalange moves in at least one of flexion or extension relative to the hub;
a second phalange coupled to the first phalange; and
a lever coupled to the first phalange, wherein the lever moves between a first position and a second position, and wherein:
in the first position, the lever is configured to prevent movement of the first phalange and the second phalange in extension; and
in the second position, the lever is configured to allow movement of the first phalange and the second phalange in extension.

2. The prosthetic finger of claim 1, wherein:
the hub defines a set of teeth;
in the first position, the lever is configured to engage with the set of teeth to prevent movement of the first phalange and the second phalange in extension; and
in the second position, the lever is configured to disengage with the set of teeth to allow movement of the first phalange and the second phalange in extension.

3. The prosthetic finger of claim 2, further comprising:
a stop coupled to the hub; and
a prop coupled to the first phalange, wherein:
the prop is configured to contact the stop, and
the prop is configured to move the lever from the first position to the second position when the prop contacts the stop.

4. The prosthetic finger of claim 3, further comprising:
a spring coupled to at least one of the first phalange or the second phalange, wherein the spring is configured to provide a biasing force that urges the first phalange and the second phalange to move in extension when the lever is in the second position.

5. The prosthetic finger of claim 1, further comprising:
a spring coupled to the first phalange and configured to provide a biasing force that urges the lever into the first position.

6. The prosthetic finger of claim 1, further comprising a collar coupled to the hub and the bone implant, wherein the hub is configured to clamp the collar to the bone implant.

7. The prosthetic finger of claim 6, wherein:
the hub further includes:
a slot,
a first protrusion extending outward from the hub and positioned on a first side of the slot, and
a second protrusion extending outward from the hub and positioned on a second side of the slot; and
the slot, the first protrusion, and the second protrusion facilitate clamping of the collar to the bone implant.

8. The prosthetic finger of claim 1, further comprising a link bar coupled to the hub and the second phalange, wherein the link bar is configured to couple motion of the first phalange to motion of the second phalange such that, when the first phalange is moved in flexion, the second phalange is also moved in flexion.

9. A prosthetic finger comprising:
a first phalange, wherein the first phalange moves in at least one of flexion or extension;
a second phalange coupled to the first phalange;
a third phalange coupled to the second phalange; and
a lever coupled to the first phalange, wherein the lever moves between a first position and a second position, and wherein:
in the first position, the lever allows movement of the first phalange, the second phalange, and the third phalange in flexion; and
in the second position, the lever allows movement of the first phalange, the second phalange, and the third phalange in extension.

10. The prosthetic finger of claim 9, further comprising a hub coupled to the first phalange, wherein:
the hub defines a set of teeth;
in the first position, the lever is configured to engage with the set of teeth to allow movement of the first phalange, the second phalange, and the third phalange in flexion; and
in the second position, the lever is configured to disengage with the set of teeth to allow movement of the first phalange, the second phalange, and the third phalange in extension.

11. The prosthetic finger of claim 10, further comprising:
a stop coupled to the hub; and
a prop coupled to the first phalange, wherein:
the prop is configured to contact the stop, and
the prop is configured to move the lever from the first position to the second position when the prop contacts the stop.

12. The prosthetic finger of claim 10, further comprising:
a collar; and
a bone implant, wherein the collar is coupled to the hub and the bone implant, and wherein the hub is configured to clamp the collar to the bone implant.

13. The prosthetic finger of claim 9, further comprising:
a hub coupled to the first phalange;
a link bar coupled to the hub and the second phalange; and
a second link bar coupled to the first phalange and the third phalange, wherein the link bar is configured to couple motion of the first phalange to motion of the second phalange, and wherein the second link bar is configured to couple motion of the first phalange to motion of the third phalange.

14. The prosthetic finger of claim 9, further comprising a spring coupled to at least one of the first phalange or the second phalange, wherein the spring is configured to provide a biasing force that urges the first phalange, the second phalange, and the third phalange to move in extension when the lever is in the second position.

15. The prosthetic finger of claim 9, further comprising a spring coupled to the first phalange and configured to provide a biasing force that urges the lever into the first position.

16. A prosthetic finger comprising:
a track that attaches to a bone implant;
a first phalange coupled to the track, wherein the first phalange moves in at least one of flexion or extension along the track;
a second phalange coupled to the first phalange; and
a lever coupled to the first phalange, wherein the lever moves between a first position and a second position, and wherein:
in the first position, the lever prevents movement of the first phalange and the second phalange in extension; and
in the second position, the lever allows movement of the first phalange and the second phalange in extension.

17. The prosthetic finger of claim 16, wherein:
the track defines a set of teeth;
in the first position, the lever is configured to engage with the set of teeth to prevent movement of the first phalange and the second phalange in extension; and
in the second position, the lever is configured to disengage with the set of teeth to allow movement of the first phalange and the second phalange in extension.

18. The prosthetic finger of claim 17, wherein, in the first position, the lever is further configured to allow movement of the first phalange and the second phalange in flexion.

19. The prosthetic finger of claim 16, further comprising a link bar coupled to the track and the second phalange, wherein the link bar is configured to couple motion of the first phalange to motion of the second phalange such that when the first phalange is moved in flexion, the second phalange is also moved in flexion.

20. The prosthetic finger of claim 16, further comprising:
a stop coupled to the track; and
a prop coupled to the first phalange, wherein:
the prop is configured to contact the stop, and
the prop is configured to move the lever from the first position to the second position when the prop contacts the stop.
